(19) United States
(12) Patent Application Publication
(10) Pub. No.: US 2018/0311299 A1
(43) Pub. Date: Nov. 1, 2018

(54) EFFICACY OF AN ANTI-C5 ANTIBODY IN THE PREVENTION OF ANTIBODY MEDIATED REJECTION IN SENSITIZED RECIPIENTS OF A KIDNEY TRANSPLANT

(71) Applicant: Alexion Pharmaceuticals, Inc., New Haven, CT (US)

(72) Inventors: Herman GRIFFIN, Waterford, CT (US); Xiang GAO, Guiford, CT (US); Rebecca VELEZ, Madison, CT (US); Wei-Jian PAN, Pullman, WA (US)

(21) Appl. No.: 15/569,338

(22) PCT Filed: Apr. 29, 2016
(86) PCT No.: PCT/US2016/030067

§ 371 Date: Oct. 25, 2017

Related U.S. Application Data
(60) Provisional application No. 62/156,007, filed on May 1, 2015.
(60) Provisional application No. 62/155,965, filed on May 1, 2015.

(51) Int. Cl. A61K 38/00; A61P 13/12; A61P 37/06; C07K 16/18; C07K 16/40

(52) U.S. Cl. CPC A61K 38/005; A61P 13/12; A61K 31/4353; C07K 16/18; C07K 16/40; A61P 37/06

(57) ABSTRACT

A method for preventing AMR in a human kidney transplant recipient is provided. The method comprises: selecting a deceased donor; selecting a kidney transplant recipient; transplanting the kidney from the donor to the recipient; and administering a therapeutically effective dose of an anti-C5 antibody or fragment thereof to the recipient. The recipient is generally sensitized to the donor. Also provided is a method for treating AMR in a kidney transplant patient. The method comprises: selecting a kidney transplant patient having symptoms of AMR; and administering a therapeutically effective dose of an anti¬ cs antibody or fragment thereof to the patient; wherein the dose of anti-C5 antibody or fragment thereof reduces the symptoms of AMR.

### TECHNICAL FIELD

This invention relates to the fields of immunology and more specifically to antibody mediated rejection.

### BACKGROUND

Solid organ transplantation remains the most effective form of therapy for treatment of patients with end-stage kidney disease. [24; the numbering herein correspond to references cited in Appendix B, List of References] In 2009, there were over 50,000 patients in Europe on the waiting list for kidney transplant; only ⅓ of these patients received a transplant. [25] While the demand for kidneys has continued to rise, the availability of organs has remained roughly the same over the last decade. Two major impediments to successful kidney transplantation are the shortage of available organs and the number of sensitized recipients.

Nearly a third of potential recipients on the Organ Procurement and Transplantation Network—UNOS renal transplant waiting-list are considered sensitized to their donor. These patients have pre-formed antibodies against an array of donor-specific human leukocyte antigens (HLA) or donor specific antibodies (DSAs). Sensitization can occur from previous exposure to donor antigens through blood transfusions, pregnancy, and/or prior organ transplantation. The presence of DSAs can lead to antibody mediated rejection (AMR) and three types have been reported:
Hyperacute rejection which presents within minutes of revascularization;
AMR which presents within days to weeks after transplantation; [26], [11]
Chronic AMR which occurs following the “de novo” generation of donor-specific antibodies and generally occurs several months to years from the time of transplant.

To date, there are no approved therapeutic agents indicated for the treatment or prevention of AMR.

### SUMMARY

This disclosure provides a method for preventing antibody mediated rejection in a deceased human kidney transplant recipient comprising the steps of: selecting a deceased donor; selecting a kidney transplant recipient, wherein the recipient is sensitized to the donor; transplanting the kidney from the donor to the recipient; and administering a therapeutically effective dose of an anti-C5 antibody, or binding fragment thereof to the recipient.

In certain other aspects, this disclosure provides a method for treating antibody mediated rejection in a kidney transplant recipient comprising: selecting a kidney transplant recipient having symptoms of antibody mediated rejection; and administering a therapeutically effective dose of an anti-C5 antibody or fragment thereof to the recipient; wherein the dose of anti-C5 antibody, or fragment thereof reduces the symptoms of antibody mediated rejection in kidney transplant recipients.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 is a schematic illustration of the study design for preventing AMR in recipients of deceased donor kidney transplants.

FIG. 2 is a graph which shows the Graft and Patient Survival Through 1 Year.

### DETAILED DESCRIPTION

### Definitions

As used herein, the word “a” or “plurality” before a noun represents one or more of the particular noun. For example, the phrase “a mammalian cell” represents “one or more mammalian cells.”

As used herein, the term “subject” or “patient” is a human patient.

As used herein the term “complement-mediated damage” refers to a pathological condition in which complement activation contributes in an observable or measurable way to the pathology of the condition. For example, complement-mediated damage can be characterized by the destruction of cells through complement activation.

As used herein the term “reducing” refers to a decrease by a statistically significant amount. For example, in one embodiment, reducing refers to either partially or completely inhibiting an activity or decreasing or lowering an activity. In one embodiment, “reducing” means a decrease by at least 10% compared to a reference level, for example a decrease by at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or up to and including a 100% decrease compared to a reference sample, or any decrease between 10-100% compared to a reference level.

As used herein the term “reducing the incidence” and “improving function” refer to a beneficial effect, e.g., amelioration or an improvement over baseline. Frequently the improvement over baseline is statistically significant. For example, “reducing the incidence” and “improving function” may refer to an amelioration of at least 10% as compared to a reference level, for example, an improvement of at least about 20%, or at least about 30%, or at least about 40%, or at least about 50%, or at least about 60%, or at least about 70%, or at least about 80%, or at least about 90% or up to and including a 100% improvement or any improvement between 10-100% as compared to a reference level, or at least about a 2-fold, or at least about a 3-fold, or at least about a 4-fold, or at least about a 5-fold, or at least about a 6-fold, or at least about a 7-fold, or at least about a 8-fold, or at least about a 9-fold, or at least about a 10-fold improvement, or any improvement between 2-fold and 10-fold or greater, as compared to a reference level.

As used herein the term “transplant” refers to the replacement of an organ, for example, a kidney, in a human or non-human animal recipient. The purpose of replacement is to remove a diseased organ or tissue in the host and replace it with a healthy organ or tissue from the donor. Where the donor and the recipient are the same species the transplant is known as an “allograft”. Where the donor and the recipient are dissimilar species the transplant is known as a “xenograft”. The techniques necessary for transplantation are varied and depend to a large extent on the nature of the organ being transplanted. The success of the transplant as a therapeutic modality depends on a number of possible physiological outcomes. For example, the host may reject the new organ via antibody-dependent hyperacute rejection mechanisms, cell-mediated acute rejection or chronic degenerative processes.

As used herein the term “reperfusion” refers to the act of restoring the flow of blood to an organ or tissue (e.g., a kidney). Reperfusion injury is the tissue damage caused when blood supply returns to the tissue after a period of ischemia or lack of oxygen. The absence of oxygen and nutrients from blood during the ischemic period creates a condition in which the restoration of circulation results in inflammation and oxidative damage through the induction of oxidative stress rather than restoration of normal function. Kidneys from deceased donors are exposed to much greater ischemic damage, as compared to living donors, before recovery and show reduced chances for proper early as well as long-term function. Kosieradzki M et al. Transplant Proc. 2008 December; 40(10):3279-88. Techniques for reperfusion of organs and tissue are well known in the art, and are disclosed in International Patent Application WO2011/002926, and U.S. Pat. Nos. 5,723,282 and 5,699,793.

The term “sensitized” used in connection with a recipient refers to a recipient that has exceptionally high antibody levels that react to foreign tissue, such as a donated organ.

As used here the term “rejection” refers to the process or processes by which the immune response of an organ transplant recipient mounts a reaction against the transplanted organ, cell or tissue, sufficient to impair or destroy normal function of the organ. The immune system response can involve specific (antibody and T cell-dependent) or non-specific (phagocytic, complement-dependent, etc.) mechanisms, or both.

The term “effective amount” refers to an amount of an agent that provides the desired biological, therapeutic, and/or prophylactic result. That result can be reduction, amelioration, palliation, lessening, delaying, and/or alleviation of one or more of the signs or symptoms of TBI or any other desired alteration of a biological system

The term “antibody” is known in the art. Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

The term “deceased donor” refers to an individual who has irreversibly lost all brain function. This may occur after an injury such as a fall, motor vehicle accident or a stroke. The determination of irreversibility, as well as the determination that all brain function is not present, are only made after repeated, confirmatory testing over a prolonged period of time.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Antibody Mediated Rejection

Over the past three decades, improvements in solid organ transplantation have paralleled advances in medical management, tissue typing, organ preservation and immunosuppression. During this time, the use of calcineurin inhibitors has focused attention on the role of T cells in allograft rejection; a form of rejection known as acute cellular rejection. More recently, AMR, a rejection reaction that results from the action of antibodies on the allograft has gained attention as a significant obstacle to successful kidney transplantation. This form of rejection causes severe and rapid dysfunction and loss of allografts. [1-4]

The most common mechanism underlying AMR is an anamnestic response that originates from previous antigenic exposure. These DSA responses are usually robust and result in the rapid production of high levels of DSA and acute allograft dysfunction. [5] The mechanism of injury in AMR involves antigens that initiate the production of DSAs resulting in antigen-antibody interactions, complement activation and inflammation, and the resultant donor tissue damage. [6]

The main target of DSA's is endothelial cells within the microcirculation of the donor organ. This leads to activation of the complement cascade, which initiates injury to the capillaries. Complement activation leads to C4d deposition in the peritubular capillaries of the renal allograft. The C4d deposition is an important diagnostic criterion for the development of AMR.

The impact of AMR on graft survival is dramatic and continues long after the initial inflammatory condition has resolved as was recently demonstrated in a study by LeFaucheur and Glotz. In this single center study of a large cohort of sensitized recipients, the investigators compared allograft survival for recipients successfully treated for AMR versus those that never experienced AMR. [9]

The effect of AMR on allograft survival, in spite of successful AMR treatment, is demonstrated by the data in Table 1 below. The data in this single center study of deceased donor kidney recipients, who were sensitized to their donors, compared the survival of the transplanted kidneys for those who experienced AMR to those who did not. The outcomes were independent of whether the recipients continued to have persistent DSA. The results strongly support the concept that prevention of the inflammatory lesion of AMR, rather than treatment intervention once AMR develops, is the key factor to transplantation across the humoral immune barrier. [9] All but two episodes of AMR occurred within six weeks, with most occurring within four weeks of transplantation, which is consistent with multiple reports in the literature by numerous investigators describing AMR as a very early clinical event. [10-12]

TABLE 1
Allograft Survival for DSA+ DD Kidney
Transplant Recipients With and Without AMR

Recipient Allograft
Survival
with and
without AMR

| | AMR+ | AMR− |
|---|---|---|
| Time Point | N = 29 | N = 54 |
| 1 year | 79.3% | 88.6% |
| 3 years | 68.9% | 88.6% |
| 8 years | 41.7% | 71.8% |

The key results from these additional reports are summarized in Table 2. Stegall, et al. described a series of 19 kidney transplant recipients who received kidney transplants following desensitization and who developed AMR. All occurrences of AMR occurred within the first six weeks and most within four weeks post-transplantation. [13] Montgomery and colleagues described another series of 62 patients in whom all instances of AMR occurred within the first 10 days post-transplantation. [10] Regardless of the clinical setting, a common theme is that most instances of AMR are reported to occur very early following transplantation.

TABLE 2
Publications on AMR in Kidney Transplantation

| | | Time |
|---|---|---|
| Author/year (reference) | Number of Patients | to Diagnosis of AMR |
| Stegall (2006)^{[13]} | 19 | <6 weeks |
| Montgomery (2004)^{[10]} | 62 (pediatric | <10 days |
| | population) | |
| Rostaing (2009)^{[14]} | 22 | Mean 21 days |
| Faguer (2007)^{[15]} | 8 | <6 weeks |
| Crespo (2001)^{[16]} | 18 | <22 days |
| White (2004)^{[17]} | 9 | <28 days |
| Braun (2004)^{[18]} | 1 | Day 7 |
| Han (2008)^{[19]} | 13 | <10 days |
| Muro (2005)^{[20]} | 1 | Day 2 |
| LeFaucheur (2010)^{[9]} | 29 | <6 weeks |
| Higgins (2009)^{[21]} | 36 | <40 days |

Taken together, this clinical experience demonstrates that AMR is a lesion that occurs early after transplantation and points to the importance of prevention of the acute inflammatory lesion of AMR during the first month post-transplantation.

Desensitization Protocols, Prophylaxis and Treatment for AMR

DSA reduction techniques (desensitization) are used to facilitate kidney transplantation for recipients who are sensitized to their donor organs by lowering the amount of circulating DSA. Techniques include direct antibody removal by plasmapheresis (PP), immune modulation using intravenous immune globulins (IVIg), and attempts to deplete B cells using a variety of immunosuppressive agents. Of all of these modalities only plasmapheresis assures immediate removal of DSA. However, plasmapheresis does not result in long-term reduction in HLA antibody. Unless a transplant is received within several days of ‘desensitization’ DSA typically return to pre-desensitization levels. As there is no way to predict when a deceased donor organ will become available, plasmapheresis is rarely part of the desensitization protocol for patients on the organ donor waiting list. [22]

There is no general consensus with regard to quantitative levels of DSA that adequately define the risk of developing AMR for any given patient but experience has demonstrated that increasing levels of DSA correlated directly with the risk to develop AMR. Lefaucher and Glotz recently reported their single center experience transplanting DSA positive, CDC crossmatch negative recipients. In that study they examined the relationship between the levels of DSA as detected by the Luminex single antigen bead technique and development of AMR. These investigators found that in recipients with MFI <3000 the prevalence of AMR was 18.7% and while it was 36.4% and 51.3% respectively for those patients with MFI between 3000 and 6000 and >6000. They also observed that kidneys having AMR had significantly shorter graft survival than did those, which did not experience AMR (9).

When AMR occurs it must be treated as early as possible to avoid irreversible damage to or loss of the transplant. Today the standard of care for AMR is plasmapheresis (PP) with or without IVIg.

Role of Complement in AMR

AMR can result from uncontrolled complement mediated injury that is initiated when DSA binds to receptors on the donor organ blood vessel endothelium. This antibody-antigen interaction results in activation of the complement cascade with the resultant production of complement split products C5a and C5b. C5a is a potent anaphylotoxin and inflammatory mediator while C5b is a necessary component for formation of the C5b-9 terminal complement complex, also known as the membrane attack complex. C5b-9 is an activator of leukocytes and vascular cells and stimulates the secretion of mediators from storage granules and the translocation of P-selectin from platelet α-granules to the plasma membrane. P-selectin initiates adhesion of monocytes and platelets to the vascular endothelium and serves as a co-stimulatory molecule for the production of inflammatory mediators. In addition, C5b-9-activated endothelial cells synthesize IL-8, tissue factor and monocyte chemotactic protein 1 (MCP-1), which is an important chemotactic factor in macrophage recruitment to sites of tissue injury.

Complement activation can be documented by measuring complement protein by-products. While some complement components bind to the antibody-antigen complex, others can be found in the local environment. For example, C4d, a stable complement component of the proximal portion of the complement cascade, can be localized by immunohistologic techniques in tissue specimens near sites of inflammation and is used as a marker for complement activation in allograft biopsy specimens.

HLA Antigens

HLA molecules are membrane bound glycoproteins that bind processed antigenic peptides and present them to T cells. The essential role of the HLA antigens lies in the control of self-recognition and thus defense against microorganisms. Based on the structure of the antigens produced and their function, there are two classes of HLA antigens, HLA Class I and Class II.

HLA Class I antigens are expressed on all nucleated cells of the body. Additionally, they are found in soluble form in plasma and adsorbed onto the surface of platelets. Erythrocytes also adsorb HLA Class I antigens.

The tissue distribution of HLA Class II antigens is confined to the “immune competent” cells, including B-lymphocytes, macrophages, and endothelial cells and activated T-lymphocytes. The expression of HLA Class II, on cells, which would not normally express them, is stimulated by cytokines like interferon-γ and is associated with acute graft rejection in the setting of transplantation.

There are important differences in HLA expression between T and B cells, which influence the interpretation of the crossmatch. T cells do not constitutively express HLA class II so the result of a T-cell crossmatch generally reflects antibodies to HLA class I only. B cells on the other hand express both HLA class I and II. Because of this, a positive B-cell crossmatch is more difficult to interpret than a positive T-cell cross match.

It may be due to antibodies directed against HLA class I or II or both. A negative B-cell crossmatch in the presence of a positive T-cell crossmatch suggests a technical error.

Transplanting in the setting of a positive T-cell crossmatch, which is not due to an autoantibody, is likely to generate a very poor outcome.

B-cell CDC crossmatching is not as predictive of hyper acute rejection (HAR) as the T-cell CDC crossmatch. B-cell crossmatches are often performed as part of the immunologic assessment before live donor transplantation when there is more time to determine the significance of the result. Paired with information about the presence of DSA, determined by more specific means such as antigen-coated beads (Luminex, discussed below) the B-cell CDC crossmatch results may be more meaningful. If a B-cell crossmatch is positive and there are no detectable antibodies to class I or II antigens, the result may be falsely positive while a positive result in the presence of detectable DSA signifies that the identified DSA may be functionally relevant in that it can activate complement, and were associated with increased risk of rejection.

Cross-Matching Techniques

Cross-matching was developed in an attempt to identify recipients who are likely to develop acute vascular rejection of a graft from a given donor. This phenomenon, HAR, is a result of preformed antibodies against the donor; referred to as donor-specific antibodies (DSA). Such antibodies are usually formed as the result of previous exposure to HLA, generally through pregnancy, blood transfusion or previous transplantation.

Preformed antibodies cause rejection by binding to HLA antigens expressed on the endothelium of vessels in the transplanted kidney, resulting in activation of the complement cascade with resultant thrombosis and infarction of the graft.

Complement-Dependent Cytotoxicity (CDC) Crossmatch

A CDC crossmatch involves placing recipient serum (potentially containing donor-specific anti-HLA antibodies) onto donor lymphocytes (containing HLA antigens). A cytotoxic reaction (deemed ‘positive’) suggests the presence of preformed DSA.

The read-out of the test is the percentage of dead cells relative to live cells as determined by microscopy. The result can thus be scored on the percentage of dead cells, with 0 correlating to no dead cells; scores of 2, 4 and 6 represent increasing levels of lysis. On this basis, a score of 2 is positive at a low level, consistent with approximately 20% lysis (generally taken as the cut-off for a positive result). A score of 8 represents all cells having lysed and indicates the strongest possible reaction. The use of a scoring system allows a semi-quantitative analysis of the strength of reaction. Another way to determine the strength of the reaction is to repeat the crossmatch using serial doubling dilutions of the recipient serum (often known as a ‘titred crossmatch’). In this way, dilutions are usually performed to 1 in 2, 4, 8, 16, 32, 64 and so on.

The Flow Crossmatching Technique

A flow crossmatch involves using the same initial base ingredients as CDC crossmatching (i.e. donor lymphocytes and recipient serum). The two are mixed and then incubating them with fluorescein-labelled antibodies against human IgG (antihuman IgG fluorescein isothiocyanate [FITC]). This fluorescein-labelled antibody will bind to all the IgG antibodies in the recipient serum. If a DSA in this serum then binds to the donor lymphocytes, it will be detectable by flow cytometry.

The read-out may be reported simply as positive or negative or can be further quantitated. Intensity of fluorescence above control, referred to as channel shifts, may be reported. Generally, a mean channel shift above 50 indicates that antibody is present and above 150 indicates a very high risk and a contraindication to renal transplant except in exceptional circumstances. Channel shifts above 300 usually correlate with a positive cytotoxic crossmatch.

Luminex Testing

Luminex testing offers significant advantages over CDC and flow crossmatch in terms of defining the HLA specificity of identified antibodies. The presence of a DSA detected by Luminex in the setting of a negative or positive CDC crossmatch appears to have prognostic importance in terms of graft survival and acute rejection risk; however, there are insufficient data to determine the significance of a DSA with a negative flow crossmatch [40, 44-46].

Positive results can then be graded as weak, moderate or strong on the basis of the degree of fluorescence of the Luminex bead array. This result can be scored as a median fluorescence index (MFI). However, Luminex bead array assays are approved only for qualitative assignment of HLA. MFI cannot directly be converted into antibody titers as the MFI simply represents a marker for the bound antibody and is affected by several factors, including antibody concentration in the serum, conformation and orientation of the antigen, and antibody avidity toward the respective antigen.

Luminex testing offers significant advantages over CDC and flow crossmatch in terms of defining the HLA specificity of identified antibodies. The presence of a DSA detected by Luminex in the setting of a negative or positive CDC crossmatch appears to have prognostic importance in terms of graft survival and acute rejection risk; however, there are insufficient data to determine the significance of a DSA with a negative flow crossmatch [40, 44-46].

Glomerular Filtration Rate

The Glomerular filtration rate (GFR) is a test used to measure how well the kidneys are working. Specifically, it estimates how much blood passes through the glomeruli each minute. Glomeruli are the tiny filters in the kidneys that filter waste from the blood. The GFR may be used to determine a patient's stage of kidney disease.

GFR is equal to the clearance rate when any solute is freely filtered and is neither reabsorbed nor secreted by the kidneys. The rate therefore measured is the quantity of the substance in the urine that originated from a calculable volume of blood. The GFR can be calculated from the following formula:

$$GFR = \frac{( Urine Concentration ) \times ( Urine Flow )}{( Plasma Concentration )}$$

The product of urine concentration and urine flow equals the mass of substance excreted during the time that urine has been collected. Dividing this mass by the plasma concentration gives the volume of plasma that the mass must have originally come from during the aforementioned period of time. The GFR is typically recorded in units of volume per time, e.g., milliliters per minute mL/min.

The estimated Glomerular filtration rate (eGFR) is used to screen for and detect early kidney damage and to monitor kidney status. It is performed by ordering a creatinine test and calculating the estimated glomerular filtration rate.

The eGFR may be calculated from serum creatine using the Chronic Kidney Disease Epidemiology Collaboration (CKD-EPI) equation.

The CKD-EPI equation, expressed as a single equation, is:

eGFR=141×min(Scr/κ,1)α×max(Scr/κ,1)−1.209×0.993Age×1.018 [if female]×1.159 [if African American].

Where Scr is serum creatinine (mg/dL), K is 0.7 for females and 0.9 for males, a is −0.329 for females and −0.411 for males, min indicates the minimum of Scr/κ or 1, and max indicates the maximum of Scr/κ or 1.

Alternatively, the estimated glomerular filtration rate may be calculated using the Modification of Diet in Renal Disease (MDRD) 7 Calculation presented below.

MDRD 7 equation (MDRD7)=170×[serum creatinine(mg/dL)]-0.999×[age]−0.176×[0.762 if patient is female]×[1.18 if patient is black]×[serum urea nitrogen concentration (mg/dL)]−0.170×[serum albumin concentration (g/dL)]0.318

A person's GFR or eGFR should be interpreted in relation to the person's clinical history and presenting conditions, utilizing Table 3.

TABLE 3
GFR and Kidney Damage

| KIDNEY | | | |
|---|---|---|---|
| DAMAGE | | | |
| STAGE | DESCRIPTION | GFR | OTHER FINDINGS |
| 1 | Normal or minimal | 90+ | Protein or albumin in |
| | kidney damage with | | urine are high, cells |
| | normal GFR | | or casts seen in urine |
| 2 | Mild decrease in | 60-89 | Protein or albumin in |
| | GFR | | urine are high, cells |
| | | | or casts seen in urine |
| 3 | Moderate decrease | 30-59 | |
| | in GFR | | |
| 4 | Severe decrease in | 15-29 | |
| | GFR | | |
| 5 | Kidney failure | <15 | |

Banff Classification of Rejection

The Banff classification characterizes five categories of renal allograft pathology: (1) AMR; (2) suspicious of acute rejection; (3) acute rejection; (4) chronic sclerosing allograft nephropathy; and (5) other-changes not considered due to rejection.

The diagnosis of AMR in renal allografts is currently based on criteria established during the Banff conference on Allograft Pathology in 2007, which include the three following cardinal features:

(1) Morphologic evidence of acute or chronic tissue injury;

(2) Immunopathological staining for C4d in peritubular capillaries;

(3) Presence of circulating antibodies to donor human lymphocyte antigen or other antigens expressed on donor endothelial cells.

It is recommended that every renal allograft biopsy should be stained for C4d. C4d staining is considered positive only when depositions are found in the peritubular capillaries.

C4d is scored semi-quantitatively in four categories:

(1) No C4d staining (0% of (peritubular) capillaries)

(2) Minimal C4d staining (0-10% of (peritubular) capillaries)

(3) Focal C4d staining (10-50% of (peritubular) capillaries)

(4) Diffuse C4d staining (>50% of (peritubular) capillaries).

Anti-C5 Antibodies

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

An exemplary anti-C5 antibody is eculizumab comprising heavy and light chains having the sequences shown in SEQ ID NOs: 10 and 11, respectively, or antigen binding fragments and variants thereof. Eculizumab (also known as Soliris@) is described in U.S. Pat. No. 6,355,245.

Eculizumab (h5G1.1-mAb solution for infusion) is a humanized monoclonal antibody with binding specificity uniquely specific for the human complement C5 protein. Comprised of 1324 amino acids with a molecular mass of approximately 148 kDa, eculizumab was derived from a murine monoclonal antibody (m5G1.1-mAb) that recognizes the human complement component C5.

Humanization of the antibody was achieved by grafting the murine antibody's complementarity determining regions (CDRs) into human antibody-derived variable heavy and light chain framework regions. The constant regions of h5G1.1-mAb include the human kappa light chain and a hybrid IgG human heavy chain. The heavy chain CH1 domain, hinge region and the first 29 amino acids of the CH2 domain were derived from human IgG2, while the remainder of the CH2 domain and the CH3 domain originated from human IgG4.

Approved by the FDA and European Medicines Agency (EMA) for the treatment of paroxysmal nocturnal hemoglobinuria and atypical hemolytic uremic syndrome, eculizumab is also being studied in other complement-mediated disorders. [30-32]

In other embodiments, the antibody comprises the heavy and light chain CDRs or variable regions of eculizumab. Accordingly, in one embodiment, the antibody comprises the CDR1, CDR2, and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises V_{H} and V_{L} regions having the amino acid sequences set forth in SEQ ID NO: 7 and SEQ ID NO: 8, respectively.

Another exemplary anti-C5 antibody is antibody BNJ441 comprising heavy and light chains having the sequences shown in SEQ ID NOs:14 and 11, respectively, or antigen binding fragments and variants thereof. BNJ441 (also known as ALXN1210) is described in PCT/US2015/019225 and U.S. Pat. No. 9,079,949. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively. In another embodiment, the antibody may comprise the heavy chain constant region of BNJ441 having the amino acid sequences set forth in SEQ ID NO: 13.

Another exemplary anti-C5 antibody is antibody BNJ421 comprising heavy and light chains having the sequences shown in SEQ ID NOs:20 and 11, respectively, or antigen binding fragments and variants thereof.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises V_{H} and V_{L} regions having the amino acid sequences set forth in SEQ ID NO:12 and SEQ ID NO:8, respectively. In another embodiment, the antibody may comprise the heavy chain constant region of BNJ421 having the amino acid sequences set forth in SEQ ID NO: 9.

Another exemplary anti-C5 antibody is the 7086 antibody described in U.S. Pat. Nos. 8,241,628 and 8,883,158. In one embodiments, the antibody may comprise the heavy and light chain CDRs or variable regions of the 7086 antibody. In another embodiment, the antibody, or a fragment thereof may comprise comprising heavy chain CDR1, CDR2 and CDR3 domains having the sequences set forth in SEQ ID NOs:21, 22, and 23, respectively, and light chain CDR1, CDR2 and CDR3 domains having the sequences set forth in SEQ ID NOs:24, 25, and 26, respectively. In another embodiment, the antibody or fragment thereof may comprise the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiments, the antibody may comprise the heavy and light chain CDRs or variable regions of the 8110 antibody. The antibody, or fragment thereof may comprise heavy chain CDR1, CDR2 and CDR3 domains having the sequences set forth in SEQ ID NOs:29, 30, and 31, respectively, and light chain CDR1, CDR2 and CDR3 domains having the sequences set forth in SEQ ID NOs:32, 33, and 34, respectively. In another embodiment, the antibody may comprise the VH region of the 8110 antibody having the sequence set forth in SEQ ID NO:35, and the V_{L} region of the 8110 antibody having the sequence set forth in SEQ ID NO:36.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627.

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance). Anti-C5 antibodies, or antigen-binding fragments thereof described herein, used in the methods described herein can be generated using a variety of art-recognized techniques.

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

Protocols

One embodiment the disclosure provides a method for preventing AMR in a human kidney transplant recipient. The method includes the steps of: selecting a deceased donor; selecting a kidney transplant recipient; transplanting the kidney from the donor to the recipient; and administering a therapeutically effective dose of an anti-C5 antibody or fragment thereof to the recipient. The recipient is generally sensitized to the donor.

Generally, the an anti-C5 antibody or fragment thereof reduces the likelihood that the recipient will develop AMR, when compared to a control group of sensitized kidney patients that did not receive the eculizumab.

The likelihood of developing AMR may be reduced by at least 9 weeks, 6 months, 12 months, 18 months, 24 months, 30 months or 36 months or more after the transplantation.

The therapeutically effective dose of eculizumab may reduce the cumulative incidence of AMR that occurs between 9 weeks and 12 months post-transplantation, when compared to the control group.

The therapeutically effective dose of eculizumab may also reduce treatment failure defined as the occurrence of: (a) biopsy proven AMR; (b) graft loss; (c) patient death; and (d) at 12 months post transplantation, when compared to the control group.

The therapeutically effective dose of eculizumab may improve the graft and patient survival at 6 months and 12 months post-transplantation, when compared to the control group.

The therapeutically effective dose of eculizumab may reduce the cumulative number of plasmapheresis treatments at 12 months post-transplantation, when compared to the control group.

The therapeutically effective dose of eculizumab may reduce the incidence of patients requiring splenectomy at 12 months post-transplantation, when compared to the control group.

The therapeutically effective dose of eculizumab may reduce the cumulative incidence and duration of dialysis between 7 days and 12 months post-transplantation, when compared to the control group.

The therapeutically effective dose of eculizumab may reduce the cumulative number of days the serum creatinine is more than 30% above its nadir following the diagnosis of AMR.

The therapeutically effective dose of eculizumab may improve the renal function between 4 weeks and 12 months post-transplantation as measured by: the estimated glomerular filtration rate calculated by Modification of Diet in Renal Disease 7 (MDRD7) on at least 3 consecutive measurements taken at least 2 days apart while not on plasmapheresis or dialysis that vary ≤20%, and serum creatinine defined as the value on at least 3 consecutive measurements that vary ≤20% taken at least 2 days apart while not on plasmapheresis or dialysis.

Generally, the therapeutically effective dose includes a 1200 mg dose on the day of the transplant, and 900 mg of eculizumab on the following post-transplantation days: 1, 7, 14 (±2 days) and 21 (±2 days). The therapeutically effective dose further usually also includes administering 1200 mg of eculizumab on the following post-transplantation weeks: week 5 (±2 days), week 7 (±2 days) and week 9 (±2 days).

Generally, on the day of the transplant the eculizumab is administered prior to reperfusion of the kidney allograft. Often the eculizumab is administered from about 30 minutes to about 3 hours prior to reperfusion of the kidney allograft. Usually, the eculizumab is administered about 1 hour prior to reperfusion of the kidney allograft.

Frequently the day 1 dose of eculizumab is administered from about 18 to about 30 hours after reperfusion of the kidney allograft. Usually, the day 1 dose is administered about 24 hours after reperfusion of the kidney allograft.

Generally, the eculizumab is maintained at plasma levels of about 50 to about 100 μg/mL.

Often, the recipient's medical history includes at least one sensitizing event selected from the group consisting of: prior solid organ or tissue allograft; pregnancy; blood transfusion; and prior exposure to the specific donor's HLA.

The recipient often has a historical positive complement-dependent cytotoxicity cross-match. The recipient may have a B cell flow cytometric cross-match from about 300 to about 500 mean channel shift. Sometimes the recipient has a T cell flow cytometric cross-match from about 300 to about 500 mean channel shift.

The recipient may have a donor specific antibody identified by a single antigen bead assay with a single mean fluorescence intensity greater than about 3000. The recipient may have a single mean fluorescence intensity from about 3000 to about 6000. Sometimes, the recipient has a single mean fluorescence intensity from about 3000 to about 12000.

Usually a diagnosis of AMR is based on the presence of circulating anti-donor specific antibodies, and morphologic evidence of acute tissue injury. The evidence of acute tissue injury may be based on a biopsy. A diagnosis of AMR may be based on the recipient exhibiting histological findings consistent with Banff Class II or III AMR on transplant biopsy.

Generally, the method of the disclosure results in the kidney allograft surviving for at least six months. The kidney allograft may survive for at least one year. Sometimes the kidney allograft survives for at least three years. The kidney allograft may survive for at least five years. The method may result in the kidney allograft surviving for the remaining life-time of the recipient.

The method of the disclosure may also include a step of administering at least one immunosuppressive drug. Generally immunosuppressive drug may be tacrolimus, mycophenolate mofetil, or prednisone.

In another embodiment the disclosure provides for treating AMR in a kidney transplant patient. The method includes selecting a kidney transplant patient having symptoms of AMR; and administering a therapeutically effective dose of eculizumab to the patient; wherein the dose of eculizumab reduces the symptoms of AMR.

Generally, the therapeutically effective dose may refer to a dosing schedule that comprises administering to the patient a 1200 mg first dose; 900 mg weekly for 4 doses (weeks 1, 2, 3, 4) and 1200 mg at week 5. The therapeutically effective dose may also include a step of administering 1200 mg of eculizumab at weeks 7 and 9.

Often, the method may include a step of administering plasmapheresis to the patient. The method may also include a step of administering immunoglobulin to the patient. In some embodiments the method may also include a step of administering both plasmapheresis and immunoglobulin to the patient.

The symptoms of AMR in the patient may include acute graft dysfunction, (elevation of creatinine above post transplant nadir) and often includes two out of three, of the following: the presence of circulating donor specific antibodies; histological findings consistent with Banff Class II or III AMR on transplant biopsy and, peritubular capillary c4d positivity on transplant biopsy.

Frequently, the patient has an increase in glomerular filtration rate at 3 months post treatment. Often, the patient has an increase in glomerular filtration rate at 12 months post treatment.

Generally, the recipient is an adult between 18 and 75 years of age.

Compositions and Formulations

The eculizumab can be administered as a fixed dose, or in a milligram per kilogram (mg/kg) dose. While in no way intended to be limiting, exemplary dosage ranges include, e.g., 1-100 μg/kg, 0.5-50 μg/kg, 0.1-100 μg/kg, 0.5-25 μg/kg, 1-20 μg/kg, and 1-10 μg/kg, 1-100 mg/kg, 0.5-50 mg/kg, 0.1-100 mg/kg, 0.5-25 mg/kg, 1-20 mg/kg, and 1-10 mg/kg. Exemplary dosages of the anti-C5 antibody, or antigen-binding fragment thereof, include, without limitation, 0.1 μg/kg, 0.5 μg/kg, 1.0 μg/kg, 2.0 μg/kg, 4 μg/kg, and 8 μg/kg, 0.1 mg/kg, 0.5 mg/kg, 1.0 mg/kg, 2.0 mg/kg, 4 mg/kg, and 8 mg/kg.

The eculizumab can be formulated as a pharmaceutical composition. The compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see e.g., Berge et al. (1977) J. Pharm. Sci. 66:1-19).

The compositions can be formulated according to standard methods. Pharmaceutical formulation is a well-established art, and is further described in, e.g., Gennaro (2000) “Remington: The Science and Practice of Pharmacy,” 20th Edition, Lippincott, Williams & Wilkins (ISBN: 0683306472); Ansel et al. (1999) “Pharmaceutical Dosage Forms and Drug Delivery Systems,” 7th Edition, Lippincott Williams & Wilkins Publishers (ISBN: 0683305727); and Kibbe (2000) “Handbook of Pharmaceutical Excipients American Pharmaceutical Association,” 3rd Edition (ISBN: 091733096X).

The pharmaceutical compositions can be in a variety of forms. These forms include, e.g., liquid solutions (e.g., injectable and infusible solutions).

Kits

The present invention also provides kits comprising the anti-C5 antibody, or antigen-binding fragment thereof, or compositions thereof (or unit dosages forms and/or articles of manufacture) and may further comprise instruction(s) on methods of use. The kits described herein may further include other materials desirable from a commercial and user standpoint, including other buffers, diluents, filters, needles, syringes, and package inserts with instructions for performing any methods described herein.

The contents of all references, Genbank entries, patents and published patent applications cited throughout this application are expressly incorporated herein by reference.

Without limiting the disclosure, a number of embodiments of the disclosure are described below for purpose of illustration.

Item 1: A method for preventing antibody mediated rejection in a human kidney transplant recipient comprising the steps of: selecting a deceased donor; selecting a kidney transplant recipient, wherein the recipient is sensitized to the donor; transplanting the kidney from the donor to the recipient; and administering a therapeutically effective dose of an anti-C5 antibody, or binding fragment thereof to the recipient.

Item 2: The method of item 1, wherein the anti-C5 antibody, or binding fragment thereof reduces the likelihood that the recipient will develop antibody mediated rejection.

Item 3: The method of items 1 and 2, wherein the therapeutically effective dose of the anti-C5 antibody, or binding fragment thereof reduces the cumulative incidence of antibody mediated rejection that occurs between 9 weeks and 12 months post-transplantation.

Item 4: The method of any of the preceding items, wherein the therapeutically effective dose of the anti-C5 antibody, or binding fragment thereof reduces the treatment failure rate defined as the occurrence of: (a) biopsy proven AMR; (b) graft loss; (c) patient death; and (d) loss to follow up at 12-months post transplantation.

Item 5: The method of any of the preceding items, wherein the therapeutically effective dose of the anti-C5 antibody, or binding fragment thereof improves the graft and patient survival at months 6 and 12-months post-transplantation.

Item 6: The method of any of the preceding items, wherein the therapeutically effective dose of the anti-C5 antibody, or binding fragment thereof reduces the cumulative number of plasmapheresis treatments at 12-months post-transplantation.

Item 7: The method of any of the preceding items, wherein the therapeutically effective dose of the anti-C5 antibody, or binding fragment thereof reduces the incidence of patients requiring splenectomy at 12-months post-transplantation.

Item 8: The method of any of the preceding items, wherein the therapeutically effective dose of the anti-C5 antibody, or binding fragment thereof reduces the cumulative incidence and duration of dialysis between 7 days and 12-months post-transplantation.

Item 9: The method of any of the preceding items, wherein the therapeutically effective dose of the anti-C5 antibody, or binding fragment thereof reduces the cumulative number of days the serum creatinine is more than 30% above its nadir following the diagnosis of antibody mediated rejection.

Item 10: The method of any of the preceding items, wherein the therapeutically effective dose of the anti-C5 antibody, or binding fragment thereof improves the renal function between 4 weeks and 12-months post-transplantation as measured by: the estimated glomerular filtration rate calculated by Modification of Diet in Renal Disease 7 (MDRD7) on at least 3 consecutive measurements taken at least 2 days apart while not on plasmapheresis or dialysis that vary ≤20%, and serum creatinine defined as the value on at least 3 consecutive measurements that vary ≤20% taken at least 2 days apart while not on plasmapheresis or dialysis.

Item 11: The method of any of the preceding items, wherein the likelihood of developing antibody mediated rejection is reduced at 9 weeks post transplantation.

Item 12: The method of any of the preceding items, wherein the likelihood of developing antibody mediated rejection is reduced at 12-months post transplantation.

Item 13: The method of any of the preceding items, wherein the likelihood of developing antibody mediated rejection is reduced at 18 months post transplantation.

Item 14: The method of any of the preceding items, wherein the likelihood of developing antibody mediated rejection is reduced at 24 months post transplantation.

Item 15: The method of any of the preceding items, wherein the likelihood of developing antibody mediated rejection is reduced at 30 months post transplantation.

Item 16: The method of any of the preceding items, wherein the likelihood of developing antibody mediated rejection is reduced at 36 months post transplantation.

Item 17: The method of any of the preceding items, wherein the therapeutically effective dose comprises a 1200 mg dose on the day of the transplant, and 900 mg of the anti-C5 antibody, or binding fragment thereof on the following post-transplantation days: day 1, 7, 14 (±2 days) and 21 (±2 days).

Item 18: The method of any of the preceding items, wherein the therapeutically effective dose further comprises administering 1200 mg of the anti-C5 antibody, or binding fragment thereof on the following post-transplantation weeks: week 5 (±2 days), week 7 (±2 days) and week 9 (±2 days).

Item 19: The method of any of the preceding items, wherein on the day of the transplant the anti-C5 antibody, or binding fragment thereof is administered prior to reperfusion of the kidney allograft.

Item 20: The method of any of the preceding items, wherein the anti-C5 antibody, or binding fragment thereof is administered from about 30 minutes to about 3 hours prior to reperfusion of the kidney allograft.

Item 21: The method of any of the preceding items, wherein the anti-C5 antibody, or binding fragment thereof is administered about 1 hour prior to reperfusion of the kidney allograft.

Item 22: The method of any of the preceding items, wherein the day 1 dose of the anti-C5 antibody, or binding fragment thereof is administered from about 18 to about 30 hours after reperfusion of the kidney allograft.

Item 23: The method of any of the preceding items, wherein the day 1 dose of the anti-C5 antibody, or binding fragment thereof is administered about 24 hours after reperfusion of the kidney allograft.

Item 24: The method of any of the preceding items, wherein the anti-C5 antibody, or binding fragment thereof is maintained at plasma levels of about 50 to about 100 μg/mL.

Item 25: The method of any of the preceding items, wherein the recipient's medical history includes at least one sensitizing event selected from the group consisting of: prior solid organ or tissue allograft; pregnancy; blood transfusion; and prior exposure to the specific donor's HLA.

Item 26: The method of any of the preceding items, wherein the recipient has a historical positive complement-dependent cytotoxicity cross-match.

Item 27: The method of any of the preceding items, wherein the recipient has a B cell flow cytometric cross-match from about 300 to about 500 mean channel shift.

Item 28: The method of any of the preceding items, wherein the recipient has a T cell flow cytometric cross-match from about 300 to about 500 mean channel shift.

Item 29: The method of any of the preceding items, wherein the recipient has a donor specific antibody identified by a single antigen bead assay with a single mean fluorescence intensity greater than about 3000.

Item 30: The method of any of the preceding items, wherein the recipient has a single mean fluorescence intensity from about 3000 to about 7000.

Item 31: The method of any of the preceding items, wherein the recipient has a single mean fluorescence intensity from about 3000 to about 6000.

Item 32: The method of any of the preceding items, wherein a diagnosis of antibody-mediated rejection is based on the presence of circulating anti-donor specific antibodies, and morphologic evidence of acute tissue injury.

Item 33: The method of any of the preceding items, wherein the evidence of acute tissue injury is based on a biopsy.

Item 34: The method of any of the preceding items, wherein the recipient exhibits histological findings consistent with Banff Class II or III antibody mediated rejection on transplant biopsy.

Item 35: The method of any of the preceding items, wherein the kidney allograft survives for at least six months.

Item 36: The method of any of the preceding items, wherein the kidney allograft survives for at least one year.

Item 37: The method of any of the preceding items, wherein the kidney allograft survives for at least three years.

Item 38: The method of any of the preceding items, wherein the kidney allograft survives for at least five years.

Item 39: The method of any of the preceding items, wherein the kidney allograft survives for the remaining life-time of the recipient.

Item 40: The method of any of the preceding items, further comprising a step of administering at least one immunosuppressive drug.

Item 41: The method of any of the preceding items, wherein at least one immunosuppressive drug is selected from the group consisting of tacrolimus, mycophenolate mofetil, and prednisone.

Item 42: A method for treating antibody mediated rejection in a kidney transplant recipient comprising the steps of: selecting a kidney transplant recipient having symptoms of antibody mediated rejection; administering a therapeutically effective dose of an anti-C5 antibody or fragment thereof to the recipient; wherein the dose of anti-C5 antibody, or fragment thereof reduces the symptoms of antibody mediated rejection in kidney transplant recipients.

Item 43: The method of item 42, wherein the therapeutically effective dose is a dosing schedule that comprises 1200 mg first dose; 900 mg weekly for 4 doses (Weeks 1, 2, 3, 4) and 1200 mg at week 5.

Item 44: The method of item 44, wherein the therapeutically effective dose further comprises a step of administering 1200 mg of the anti-C5 antibody or antigen-binding fragment at weeks 7 and 9.

Item 45: The method of any of items 42-44, further comprising a step of administering plasmapheresis to the recipient.

Item 46: The method of any of items 42-45, further comprising a step of administering immunoglobulin to the recipient.

Item 47: The method of any of items 42-46, further comprising a step of administering plasmapheresis and immunoglobulin to the recipient.

Item 48: The method of any of items 42-47, wherein the recipient is an adult renal transplant recipient between 18 and 75 years of age.

Item 49: The method of any of items 42-48, wherein the symptoms of antibody mediated rejection include acute graft dysfunction, (elevation of creatinine above post transplant nadir) and two out of three, of the following inclusion criteria: presence of circulating donor specific antibodies; histological findings consistent with Banff Class II or III antibody mediated rejection on transplant biopsy or, peritubular capillary c4d positivity on transplant biopsy.

Item 50: The method of any of items 42-49, wherein the recipient has an increase in glomerular filtration rate at 3 months post treatment.

Item 51: The method of any of items 42-50, wherein the patient has an increase in glomerular filtration rate at 12-months post treatment.

Item 52: The method of any of the preceding items wherein the anti-C5 antibody, or antigen binding fragment thereof, comprises CDR1, CDR2, and CDR3 heavy chain sequences as set forth in SEQ ID NOs: 1, 2, and 3, respectively, and CDR1, CDR2, and CDR3 light chain sequences as set forth in SEQ ID NOs: 4, 5, and 6, respectively.

Item 53: The method of any of the preceding items wherein the anti-C5 antibody, or antigen binding fragment thereof, comprises the VH domain having the sequence set forth in SEQ ID NO:7, and the VL domain having the sequence set forth in SEQ ID NO:8, respectively.

Item 54: The method of any of the preceding items wherein the anti-C5 antibody, or antigen binding fragment thereof, comprises a heavy chain constant region having the amino acid sequences set forth in SEQ ID NO: 9.

Item 55: The method of any of the preceding items wherein the anti-C5 antibody, or antigen binding fragment thereof, comprises the entire heavy chain and light chains having the amino acid sequences set forth in SEQ ID NO: 10 and SEQ ID NO: 11, respectively.

Item 56: The method of any of the preceding items wherein the anti-C5 antibody, or antigen binding fragment thereof, comprises CDR1, CDR2, and CDR3 heavy chain sequences as set forth in SEQ ID NOs:19, 18, and 3, respectively, and CDR1, CDR2, and CDR3 light chain sequences as set forth in SEQ ID NOs: 4, 5, and 6, respectively.

Item 57: The method of any of the preceding items wherein the anti-C5 antibody, or antigen binding fragment thereof comprises the VH domain having the sequence set forth in SEQ ID NO:12, and the VL domain having the sequence set forth in SEQ ID NO:8, respectively.

Item 58: The method of any of the preceding items wherein the anti-C5 antibody, or antigen binding fragment thereof comprises a heavy chain constant region having the amino acid sequences set forth in SEQ ID NO: 13.

Item 59: The method of any of the preceding items wherein the anti-C5 antibody, or antigen binding fragment thereof comprises the entire heavy chain and light chains having the amino acid sequences set forth in SEQ ID NO: 14 and SEQ ID NO: 11, respectively.

Item 60: The method of any of the preceding items wherein the anti-C5 antibody, or antigen binding fragment thereof comprises the entire heavy chain and light chains having the amino acid sequences set forth in SEQ ID NO: 20 and SEQ ID NO: 11, respectively, wherein administering the antibody, or antigen binding fragment thereof, decreases the risk of the patient developing DGF, compared to the absence of therapy.

Item 61: The method of any of the preceding items wherein the anti-C5 antibody, or antigen binding fragment thereof comprises CDR1, CDR2, and CDR3 heavy chain sequences as set forth in SEQ ID NOs:21, 22, and 23, respectively, and CDR1, CDR2, and CDR3 light chain sequences as set forth in SEQ ID NOs: 24, 25, and 26, respectively.

Item 62: The method of any of the preceding items wherein the anti-C5 antibody, or antigen binding fragment thereof comprises the VH domain having the sequence set forth in SEQ ID NO:27, and the VL domain having the sequence set forth in SEQ ID NO:28.

Item 63: The method of any of the preceding items wherein the anti-C5 antibody, or antigen binding fragment thereof comprises CDR1, CDR2, and CDR3 heavy chain sequences as set forth in SEQ ID NOs:29, 30, and 31, respectively, and CDR1, CDR2, and CDR3 light chain sequences as set forth in SEQ ID NOs: 32, 33, and 34, respectively.

### EXAMPLES

For this invention to be better understood, the following examples are set forth. These examples are for purposes of illustration only and are not be construed as limiting the scope of the invention in any manner.

### Example 1: An Open-Label, Single-Arm, Multicenter Trial to Determine Safety and Efficacy of Eculizumab in the Prevention of AMR in Sensitized Recipients of a Kidney Transplant from a Deceased Donor

Study Objectives and Purpose

A phase II study was conducted to assess the effect of eculizumab on the incidence of AMR, patient survival, graft survival, and loss to follow-up, in kidney transplant recipients who were sensitized to their deceased donors.

Primary Objective

The primary objective of this study was to evaluate the safety and efficacy of eculizumab to prevent AMR in sensitized recipients of deceased donor kidney transplants.

Methods

Sensitized kidney transplant recipients had current DSA greater than 3000 MFI as detected by SAB; or B-cell or T-cell flow cytometric crossmatch 2300 and 500 mean channel shift; or historical positive complement-dependent cytotoxicity crossmatch to donor HLA. All recipients received eculizumab 1200 mg postoperative day 0 prior to reperfusion, 900 mg on postoperative day 1, 7, 14, and 28, and 1200 mg at weeks 5, 7, 9. Rabbit ATGa was used for induction and corticosteroids, tacrolimus, and mycophenolate for maintenance immunosuppression. Post-transplant plasmapheresis was not allowed. The primary composite endpoint was clinically significant, biopsy-proven aAMR grade II/III (Banff 2007), (based on centrally read biopsy), death, graft loss, or loss to follow-up at 9 weeks post-transplant.

Results

Eighty candidates were transplanted (48 F, 32 M); median age 52 years (range, 24-70). 9/80 kidney transplant recipients met the 9 week composite endpoint based on local biopsies (11.3% [95% CI 5.3%, 20.3%]). 5 of the 9 kidney transplant recipients had AMR (6.3%) compared to 304 expected for historical controls. Graft survival at 6 and 12 months was 93.7% and 87.1%, respectively; patient survival at 6 and 12 months was 97.4%. Mean creatinine levels (mg/dL) at baseline, 1 and 12 month post-transplant were, 7.44 (±2.52), n=78; 1.86 (±1.07), n=74; and 1.80 (±1.11), n=45, respectively. No new safety signals were identified.

Conclusions

Eculizumab was effective at reducing the incidence of AMR in sensitized deceased donor kidney transplant recipients. Patient and graft survival and kidney function at 1 year were similar to those expected for nonsensitized kidney transplant recipients. Eculizumab was well tolerated.

A table of abbreviations and specialist terms is presented in Table 4.

TABLE 1
Abbreviations and Specialist Terms

| Abbreviation or Specialist | |
|---|---|
| Term | Explanation |
| ABO | A, B and O Blood Glycoproteins (Blood |
| | Type) |
| AE | Adverse Event |
| aHUS | Atypical Hemolytic Uremic Syndrome |
| ALT | Alanine aminotransferase (SGPT) |
| AMR | Antibody-Mediated Rejection |
| AP | Alkaline Phosphatase |
| AST | Aspartate aminotransferase (SGOT) |
| BFXM | B-Cell Cytometric Flow Crossmatch |
| BE | Bioequivalence |
| BID | Twice Daily |
| BK | BK Virus |
| BUN | Blood Urea Nitrogen |
| ° C. | Degrees Celsius |
| CBC | Complete Blood Count |
| CI | Confidence Interval |
| CDC | Complement-Dependent Cytotoxicity |
| CDRs | Complementarity Determining Regions |
| CMV | Cytomegalovirus |
| CRF | Case Report Form |
| CsA | Cyclosporine |
| CyP | Cyclophosphamide |
| DD | Deceased Donor |
| DGF | Delayed Graft Function |
| EMC | Data Monitoring Committee |
| DSA | Donor Specific Antibody |
| ECG | Electrocardiogram |
| eGFR | Estimated Glomerular Filtration Rate |
| ELISA | Enzyme Linked Immunosorbent Assay |
| EMA | European Medicines Agency |
| ESRD | End-Stage Renal Disease |
| ° F. | Degrees Fahrenheit |
| FCXM | Flow Cytometric Crossmatch |
| FDA | Food and Drug Administration |
| FFP | Fresh Frozen Plasma |
| FSH | Follicle-Stimulating Hormone |
| GCP | Good Clinical Practice |
| GGT | Gamma-Glutamyltransferase |
| β-HCG | Beta-Human Chorionic Gonadotrophic |
| | Hormone |
| HBV | Hepatitis B Virus |
| HCT | Hematocrit |
| HCV | Hepatitis C Virus |
| HEENT | Head, Ears, Eyes, Nose, Throat |
| Hgb | Hemoglobin |
| HIV | Human immunodeficiency Virus |
| HLA | Human Leukocyte Antigen |
| ICF | Informed Consent Form |
| ICH | International Conference on |
| | Harmonization |
| IEC | Independent Ethics Committee |
| IgG | Immunoglobulin G |
| INR | International Normalized Ratio |
| IRB | Institutional Review Board |
| IV | Intravenous |
| IVIG | Intravenous Immune Globulin |
| kDa | Kilodalton |
| LD | Live Donor |
| LDH | Lactate Dehydrogenase |
| LF | Leflunomide |
| mAb | Monoclonal Antibody |
| MAC | Membrane Attack Complex |
| MCH | Mean Corpuscular Hemoglobin |
| MCHC | Mean Corpuscular Hemoglobin |
| | Concentration |
| MCS | Mean Channel Shift |
| MCV | Mean Corpuscular Volume |
| MDRD7 | Modification of Diet in Renal Disease 7 |
| MedDRA | Medical Dictionary for Regulatory |
| | Activities |
| MFI | Mean Fluorescence Intensity |
| MHC | Major Histocompatibility Complex |
| MMF | Mycophenolate Mofetil |
| PCP | Pneumocystis carinii/Pneumocystis |
| | jiroveci Pneumonia |
| PD | Pharmacodynamics |
| PK | Pharmacokinetics |
| Plts | Platelets |
| PP | Plasmapheresis |
| POD | Post-operative Day |
| PNH | Paroxysmal Nocturnal Hemoglobinuria |
| PRA | Percent Reactive Antibody |
| PT | Prothrombin Time |
| PTT/aPTT | Partial Thromboplastin Time/activated |
| | Partial Thromboplastin Time |
| RBC | Red Blood Cells |
| SAE | Serious Adverse Event |
| SAB | Single-bead Antigen |
| SCr | Serum Creatinine |
| SOC | Standard of Care |
| TAC | Tacrolimus |
| TEAE | Treatment Emergent Adverse Event |
| TFXM | T-Cell Cytometric Flow Crossmatch |
| XM | Crossmatch |
| WBC | White Blood Cells |

Investigational Plan

Overall Study Design

The eculizumab study was an open-label, single-arm, multicenter, phase II study. After appropriately screened patients were cleared for transplant by the Principal Investigator, they were enrolled in the study and underwent eculizumab therapy. Patients received eculizumab (study drug) for 9 weeks post-transplantation. All patients received standard immunosuppression, prophylactic medications and post-transplantation care. The diagnosis of AMR for the determination of the primary end point was based on “for cause” kidney biopsies. In addition, protocol biopsies were performed on all patients at predetermined time points. All patients were screened for standard laboratory values, DSA titers, TFXM, BFXM, complement-dependent cytotoxicity (CDC), estimated glomerular filtration rate (eGFR) and other clinical and laboratory parameters for evaluation of primary and secondary endpoints as well as safety. The primary analysis of the data occurred after all patients had reached month 12 post-transplantation. However, patients had additional follow up at months 18, 24 and 36 post-transplantation to assess patient and graft survival, kidney disease and disease status.

Study Period (Years)

This study was estimated to require approximately 4 years for completion. The following were the expected major timelines for this study: estimated date first patient enrolled: 2^{nd} Q2012; estimated date last patient, first visit: 2^{nd} Q2014; estimated date last patient last visit: 2^{nd} Q2015; estimated date of last patient completing 3 year follow up data collection: 2^{nd} Q2017.

It was estimated that approximately 20 kidney transplant centers in Europe and Australia would be required in order to fully enroll the study. Additional sites/countries would be considered if necessary.

Pre-Screening, Screening and Enrollment

Pre-Screening:

Investigators pre-screened highly sensitized patients on their transplant waiting list in order to identify those patients that suitable for the study protocol.

Screening/Enrollment:

Patients who were considered candidates to receive a deceased donor kidney transplant by the investigative sites' selection criteria and who were sensitized to their deceased donor as defined below, were considered for enrollment in this study. Candidates for enrollment signed an informed consent form (ICF) and underwent the baseline HLA screening at the investigative site's local laboratory with duplicate specimens being sent to the central laboratory for confirmation. The local laboratory specimen values were utilized for verification that the candidates met enrollment criteria for study entry. If all inclusion criteria and none of the exclusion criteria were met, patients were vaccinated against N. meningitidis. Furthermore, all patients that were not already vaccinated within the time period of active coverage specified by the vaccine manufacturer, were re-vaccinated 30 days after initial vaccination. Tetravalent conjugated vaccines for N. meningitidis was used. If patients were not already vaccinated at least 14 days prior to receiving the first dose of eculizumab, they received prophylactic treatment with an appropriate antibiotic for 14 days after the vaccination.

Baseline parameters to determine if patients were “sensitized” include the following: sensitizing event (history of prior exposure to HLA): (a) Prior solid organ or tissue allograft; (b) Pregnancy; (c) Blood transfusion; (d) Prior exposure to specific donor's HLA.

If a patient's medical history was consistent with DSA exposure then the following blood draws were performed to confirm candidacy. Patients must have had (a) a historical positive complement-dependent cytotoxicity (CDC)(patients must have been CDC negative at time of transplant and/or (b) the B cell flow cytometric cross match (BFXM) or T cell flow cytometric cross match (TFXM) 300 and 500 mean channel shift (mcs). No patient was either a BFXM or TFXM >500 mcs and/or (c) DSA identified by single antigen bead assay (Luminex Labscreen assay) with a single MFI >3000.

The local Laboratory specimens could be used to select patients for study entry.

A duplicate set of samples were analyzed at the Central HLA laboratory.

Primary Endpoint

The primary composite endpoint was the Week 9 post-transplantation treatment failure rate defined as the occurrence of 1) biopsy-proven AMR, 2) graft loss, 3) patient death, or 4) loss to follow-up.

A diagnosis of AMR was based on kidney allograft dysfunction and a biopsy performed “for cause.” The histological diagnosis was based on Banff 2007 criteria for AMR as determined by the Central Pathology Laboratory. For this study only level II and level III AMR were accepted as defined as follows: (1) The presence of circulating anti-DSAs, and (2) morphologic evidence of acute tissue injury, such as (Type/Grade): (a) Banff 2007 level II—Capillary and/or glomerular inflammation (ptc/g>0) and/or thromboses; (b) Banff 2007 level III—Arterial-v3

Secondary Endpoints

Secondary endpoints for this study included the following: (1) the cumulative incidence of AMR that occurred between week 9 and month 12 post-transplantation (AMR of any grade that met the Banff 2007 criteria); (2) the treatment failure rate defined as the occurrence of: (a) biopsy proven AMR; (b) graft loss; (c) patient death; and (d) loss to follow up at Month 12 post transplantation; (3) the graft and patient survival at Months 6 and 12 months post-transplantation; (4) histological evidence of AMR on protocol biopsies without other clinical findings at Day 14, and Months 3 and 12 post-transplantation; (5) Characterize the overall pathological changes, including chronic AMR, on protocol biopsies at Day 14 and Months 3 and 12 post-transplantation; (6) the cumulative number of plasmapheresis treatments at 12 months post-transplantation; (7) the cumulative incidence of patients requiring splenectomy at 12 months post-transplantation; (8) the incidence of delayed graft function (DGF) post-transplantation (defined as the requirement for dialysis within the first post-transplantation week for reasons other than post-operative hyperkalemia, acute pulmonary edema or fluid overload due to comorbid conditions); (9) the cumulative incidence and duration of dialysis between 7 days and 12 months post-transplantation; (10) the cumulative number of days the serum creatinine is more than 30% above nadir following the diagnosis of AMR; (11) stable renal function between week 4 and month 12 post-transplantation as measured by: (a) the Estimated Glomerular Filtration Rate (calculated) by Modification of Diet in Renal Disease 7 (MDRD7) on at least 3 consecutive measurements taken at least 2 days apart while not on plasmapheresis or dialysis that vary ≤20%, and (b) serum creatinine defined as the value on at least 3 consecutive measurements that vary ≤20% taken at least 2 days apart while not on plasmapheresis or dialysis.

Number of Patients

An estimated 80 patients were enrolled into the single-arm study. This was based on a single-arm exact binomial test for the primary efficacy endpoint variable. See Statistics and Data Analysis section for additional details.

Treatment Assignment and Duration of Treatment

Patients who were CDC negative, and were cleared for transplantation by the Principal Investigator were enrolled and received eculizumab treatment. Patients were followed for primary and secondary endpoints to month 12 post-transplantation, and for DSA, kidney function and patient and graft survival up to month 36 post-transplantation.

Patients that were diagnosed with biopsy proven AMR during the first 9 weeks of treatment were considered treatment failures. Investigators were allowed to continue treatment of the AMR with eculizumab in addition to other agents. In addition, for biopsy proven AMR that was diagnosed after 9 weeks, investigators were permitted to also use eculizumab as part of the AMR treatment regimen. See below for dosing instructions for eculizumab.

Eculizumab Treatment

All doses of eculizumab were IV as a continuous infusion over 25-45 minutes. Patients were a negative CDC cross match prior to transplantation. Treatment started during the transplantation procedure and continued as follows: (1) eculizumab 1200 mg (4 vials) administered in the operating room approximately 1 hour prior to kidney allograft reperfusion (Day 0); (2) eculizumab 900 mg (3 vials) on the following post-transplantation days: Day 1, Day 7, Day 14 (±2 days), Day 21 (±2 days), and Day 28 (±2 days); (3) eculizumab 1200 mg (4 vials) was given on the following post-transplantation weeks: week 5 (±2 days); week 7 (±2 days); Week 9 (±2 days). PP and/or intravenous immune globulin was used only to treat biopsy proven AMR. In this setting the study drug continued to be administered per the guidelines below.

Dose Adjustment Criteria

Eculizumab was administered intravenously as a fixed dose depending upon the time relative to the transplant.

Safety Criteria for Adjustment or Stopping Doses

If an adverse reaction occurred during the administration of eculizumab, the infusion may have been slowed or stopped at the discretion of the Principal Investigator. If the infusion was slowed, the total infusion time would not exceed two hours. The adverse reaction was recorded on the AE page of the CRF.

The patients were monitored for at least one hour following completion of the infusion for signs or symptoms of an infusion reaction.

Infusion Reactions

As with all protein products, administration of eculizumab may have resulted in infusion reactions, including anaphylaxis or other hypersensitivity reactions. Eculizumab administration was interrupted in all patients experiencing severe infusion reactions and appropriate medical therapy administered. The infusion reaction must be recorded on the AE page of the CRF.

Criteria for Study Termination

The Sponsor was permitted to terminate the study at any time for safety or administrative reasons.

The Data Monitoring Committee (DMC) was in charge of monitoring the risk-benefit ratio for the patients and could make the following recommendation to Sponsor: (1) continued enrollment and dosing of eculizumab treatment; (2) enrollment at a reduced dose of eculizumab treatment; (3) increased monitoring of patients of eculizumab treatment.

Study Procedures

General Information

Transplant recipients were cared for according to the investigative site's SOC protocols employed for post-transplantation follow-up. The Principal Investigator at each site was directly responsible for supervising the care of these recipients during the length of the study.

Laboratory Information

Sites utilized local laboratories for the following tests: (1) hematology panel; (2) chemistry panel; (3) urinalysis; (4) spot urine for urine protein/creatinine ratio; (5) tacrolimus troughs; (6) activated partial prothromplastin time (aPTT), PT (Prothrombin Time), and International Normalized Ratio (INR); (7) eGFR (MDRD 7); (8) Serum Pregnancy Test for Women of Childbearing Potential; (9) BFXM and TFXM for routine management (Local [optional] and Central Laboratories [mandatory]); (10) CDC (Local and Central Laboratories); (11) the DSA by Luminex LabScreen (Local and Central Laboratories)

Central Laboratory Information

A Central Laboratory was responsible for BFXM, TFXM, CDC and DSA by Luminex LabScreen taken at predetermined times as described herein.

PK/PD samples were forwarded by the sites to the Central Laboratory for accessioning and storage until the end of the study at which time all samples were be forwarded to the sponsor for analysis.

Central Pathology Information

All protocol and “for cause” kidney biopsies were processed and analyzed by the site's Local Pathology Laboratory. Processed slides and two paraffin embedded unstained slides were also forwarded to the Central Pathology imaging center for review by a panel of independent pathologists.

Clinical Assessments

Clinical assessments were conducted routinely during the post-operative period according to the transplant site protocol and also at various time points throughout the study. These assessments included an assessment of the patient's health status, renal function and new diagnoses.

Female Patients of Child-Bearing Potential

Female candidates who were of child-bearing potential must have had a negative pregnancy test (serum beta-hCG) and practice a medically approved contraceptive regimen during the post-transplantation period for at least 5 months following discontinuation of eculizumab.

Female patients were exempt from contraception requirement if they were post-menopausal for at least 1 year before dosing or are surgically sterile (i.e., no uterus or no ovaries). Females who had their fallopian tubes banded, tied or cut were not considered surgically sterile without FSH level confirmation. Of note, females with end stage renal disease (ESRD) could be amenorrheic prior to transplantation.

Timing of Visits and Missed Visits

The schedule for clinical assessments during the Pre-Transplant, Immediate Post-Transplant, Extended Post-Transplant, and Long Term Outcome Phases are located in Tables 5, 6, 7, and 8. For practical logistical reasons the assigned visit windows were designed to allow more flexibility after the initial 9 weeks of the study.

In all cases, if a study visit was missed it was expected that a protocol deviation was documented on the appropriate forms.

Screening/Enrollment Phase

The following procedures were performed during the screening period:

Pre-Transplant Day −1 to Day 0 Prior to Transplant

Informed consent; demographics; medical history (including current medications); complete physical exam including vital signs, height and weight; determination of eligibility based on inclusion/exclusion criteria; 12-lead electrocardiogram (ECG); hematology panel; chemistry panel; urinalysis; aPTT, PT and INR; serum pregnancy test for women of childbearing potential; BFXM and TFXM for routine management (samples to Local [optional] and Central Laboratories [mandatory]); CDC (samples to Local and Central Laboratories); DSA by Luminex LabScreen (samples to Local and Central Laboratories); record concomitant medications, and assessment of AEs.

Vaccination Against N. meningitidis.

Patients were vaccinated at least 14 days prior to receiving the first dose of eculizumab or were vaccinated and received prophylactic treatment with an appropriate antibiotic for 14 days after the vaccination. Furthermore, all patients that were not already vaccinated within the time period of active coverage specified by the vaccine manufacturer, were re-vaccinated 30 days after initial vaccination. Patient were instructed on the signs and symptoms of N. meningitidis. Identification cards were provided to the patients explaining that the patients were participating in a clinical trial with a description of the Investigational Product and emergency contact information.

Enrollment

Entry criteria for the study were determined by Local Laboratory data for DSA, CDC, BFXM, and/or TFXM at Screening.

All patients were CDC negative at the time of transplant.

Immediate Post-Transplant Phase.

The Local Laboratory specimen data for BFXM, TFXM, and/or DSA were used for patient management.

During the study, patients carried a detailed card describing the “alert” symptoms for Neisseria meningitides at all times. Development of the “alert” symptoms card was the responsibility of the sponsor or its designee. The triggers for seeking immediate medical attention were any of the following symptoms: (1) headache with nausea or vomiting; (2) headache with fever; (3) headache with a stiff neck or back; (4) fever of 103° F. (39.4° C.) or higher; (5) fever and a rash; (6) confusion; (7) severe myalgia with flu-like symptoms; or (8) sensitivity to light.

Week 0 (On Transplant Day 0)

For all patients, the following were completed on the day of the transplant following the transplant: kidney transplant procedure; complete physical exam including vital signs and weight; hematology panel; chemistry panel; urinalysis; aPTT, PT and INR; BFXM and TFXM for routine management (samples to Local [optional] and Central Laboratories [mandatory]); DSA by Luminex LabScreen (samples to Local and Central Laboratories); assessment of renal function/need for dialysis; kidney allograft biopsy (post-reperfusion; send duplicate slides to Central Pathology); recorded concomitant medications; record immunosuppressive medications; assessment of AEs; administered eculizumab, 1200 mg (4 vials), approximately one hour prior to reperfusion of kidney allograft.

Baseline and peak PK and PD collection (baseline samples were taken 5-90 minutes prior to study drug infusion; peak samples were to be taken 60 minutes after the completion of the study drug infusion).

Post-Transplant Day 1

For all patients, the following were completed one day post-transplantation: abbreviated physical exam including vital signs and weight; clinical assessment including evaluation for rejection; Hematology panel; chemistry panel; aPTT, PT and INR; tacrolimus trough; BFXM and TFXM for routine management (samples to Local [optional] and Central Laboratories [mandatory]; DSA by Luminex LabScreen (samples to Local and Central Laboratories; assess renal function/need for dialysis; record concomitant medications; record immunosuppressive medications; assessment of AEs; administered eculizumab, 900 mg (3 vials); trough and peak PK and PD collected (trough samples should be taken 5-90 minutes prior to study drug infusion; peak samples are to be taken 60 minutes after the completion of the study drug infusion).

Post-Transplant Days 2-6

For all patients the following were completed: abbreviated physical exam including vital signs and weight; clinical assessment including evaluation for rejection; hematology panel; chemistry panel; aPTT, PT and INR; tacrolimus trough; assessment of renal function/need for dialysis; recorded concomitant medications; recorded immunosuppressive medications; and assessment of AEs.

Post-Transplant Day 7

For all patients the following were completed: abbreviated physical exam including vital signs and weight; clinical assessment including evaluation for rejection; hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]); DSA by Luminex LabScreen (samples to Local and Central Laboratories); assess renal function/need for dialysis; record concomitant medications; record immunosuppressive medications; assessed AEs; administered eculizumab, 900 mg (3 vials); trough and peak PK and PD collection (trough samples should were obtained 5-90 minutes prior to study drug infusion; peak samples were obtained 60 minutes after the completion of the study drug infusion).

Extended Post-Transplant Phase

All patients continued to be seen for study visits at regular intervals Post-Transplant Day 14 through Month 12 (primary efficacy analysis). The Local Laboratory specimen data for BFXM, TFXM, and/or DSA was used for patient management.

Post-Transplant Day 14/Week 2

For all patients, the following were completed: abbreviated physical exam including vital signs and weight; clinical assessment including evaluation for rejection; hematology panel; chemistry panel; aPTT, PT and INR; tacrolimus trough; BFXM and TFXM for routine management (samples to Local [optional] and Central Laboratories [mandatory]); DSA by Luminex LabScreen (samples to Local and Central Laboratories); assessed renal function/need for dialysis; kidney allograft biopsy (send duplicate slides to Central Pathology); recorded concomitant medications; recorded immunosuppressive medications; assessed AEs; administered eculizumab, 900 mg (3 vials).

Trough and peak PK and PD collection (trough samples were taken 5-90 minutes prior to study drug infusion; peak samples were to be taken 60 minutes after the completion of the study drug infusion).

Post-Transplant Day 21/Week 3

For all patients, the following were completed: abbreviated physical exam including vital signs and weight; clinical assessment including evaluation for rejection;

Hematology panel; chemistry panel; aPTT, PT and INR; Tacrolimus trough; BFXM and TFXM for routine management (samples to Local [optional] and Central Laboratories [mandatory]); DSA by Luminex LabScreen (samples to Local and Central Laboratories); assessment of renal function/need for dialysis; Recorded concomitant medications; recorded immunosuppressive medications; assessment of AEs; administration of eculizumab, 900 mg (3 vials)—No PK/PD assessments required for this dose.

Post-Transplant Day 28/Week 4

For all patients, the following were completed: abbreviated physical exam including vital signs and weight; clinical assessment including evaluation for rejection; hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]); DSA by Luminex LabScreen (samples to Local and Central Laboratories); eGFR (MDRD 7); vaccination against N. meningitides (only for patients that were not already vaccinated within the time period of active coverage specified by the vaccine manufacturer); assessment renal function/need for dialysis; record concomitant medications; recorded immunosuppressive medications; assessment of AEs; administration of eculizumab, 900 mg (3 vials);

Trough and peak PK and PD collection (trough samples should were obtained 5-90 minutes prior to study drug infusion; peak samples were to obtained 60 minutes after the completion of the study drug infusion).

Post-Transplant Days 35 and 49/Weeks 5 and 7

For all patients, the following were completed: Abbreviated physical exam including vital signs and weight; Clinical assessment including evaluation for rejection; Scr and Bu; tacrolimus trough; Assessment renal function/need for dialysis; recorded concomitant medications; recorded immunosuppressive medications; assessment of AEs; administration of eculizumab, 1200 mg (4 vials); trough and peak PK and PD collection (trough samples were obtained 5-90 minutes prior to study drug infusion; peak samples were obtained 60 minutes after the completion of the study drug infusion).

Post Transplant Day 56/Week 8

For all patients, the following were completed: abbreviated physical exam including vital signs and weight; clinical assessment including evaluation for rejection; hematology panel; chemistry panel; aPTT, PT and INR; tacrolimus trough; assessment of renal function/need for dialysis; eGFR (MDRD 7); recorded concomitant medications; recorded immunosuppressive medications; and assessment of AEs.

Post Transplant Day 63/Week 9

For all patients, the following were completed: abbreviated physical exam including vital signs and weight; clinical assessment including evaluation for rejection; hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]); DSA by Luminex LabScreen (samples to Local and Central Laboratories); assessment renal function/need for dialysis; eGFR (MDRD 7); recorded concomitant medications; recorded immunosuppressive medications; assessment of AEs; administration of eculizumab, 1200 mg (4 vials);

Trough and peak PK and PD collection (trough samples were obtained 5-90 minutes prior to study drug infusion; peak samples are to be taken 60 minutes after the completion of the study drug infusion).

Post Transplant Week 12/Month 3

For all patients, the following were completed: complete physical exam including vital signs and weight; clinical assessment including evaluation for rejection; hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]; DSA by Luminex LabScreen (samples to Local and Central Laboratories); assessment of renal function/need for dialysis; eGFR (MDRD 7); kidney allograft biopsy (sent duplicate slides to Central Pathology); record concomitant medications; recorded immunosuppressive medications; and assessment of AEs.

Post Transplant Weeks 17 & 21/Months 4 & 5

For all patients the following were completed: abbreviated physical exam including vital signs and weight; clinical assessment including evaluation for rejection; sCr and BUN; tacrolimus trough; assessment of renal function/need for dialysis; recorded concomitant medications; recorded immunosuppressive medications; assessment of AEs.

Post Transplant Week 26/Month 6

For all patients, the following were completed: complete physical exam including vital signs and weight; clinical assessment including evaluation for rejection; Hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]); DSA by Luminex LabScreen (samples to Local and Central Laboratories); assessment of renal function/need for dialysis; eGFR (MDRD 7); recorded concomitant medications; Record immunosuppressive medications; and assessment of AEs.

Post Transplant Weeks 30 & 34/Months 7 & 8

For all patients, the following were completed: abbreviated physical exam including vital signs and weight; clinical assessment including evaluation for rejection; SCr and BUN; tacrolimus trough; assessment of renal function/need for dialysis; recorded concomitant medications; recorded immunosuppressive medications; and assessment of AEs.

Post Transplant Week 38/Month 9

For all patients, the following were completed: abbreviated physical exam including vital signs and weight; clinical assessment including evaluation for rejection; hematology panel; chemistry panel; aPTT, PT and INR; tacrolimus trough; assess renal function/need for dialysis; eGFR (MDRD 7); recorded concomitant medications; recorded immunosuppressive medications; and assessment of AEs.

Post Transplant Week 52/Month 12—Study Primary Analysis Time Point

For all patients, the following were completed: completed physical exam including vital signs and weight; clinical assessment including evaluation for rejection; hematology panel; chemistry panel; urinalysis; spot urine for urine protein/creatinine ratio; aPTT, PT and INR; tacrolimus trough; BFXM and TFXM for routine management (samples to Local [optional] and Central Laboratories [mandatory]); DSA by Luminex LabScreen (samples to Local and Central Laboratories); assess renal function/need for dialysis; eGFR (MDRD 7); kidney allograft biopsy (send duplicate slides to Central Pathology); recorded concomitant medications; recorded immunosuppressive medications; and assessment of AEs.

Long Term Outcomes Phase

Additional study visits occurred at Months 18, 24 and 36 for long term follow up data. This data was not used for purposes of the primary efficacy analysis.

Post Transplant Months 18 and 24

For all patients, the following were completed: assessment of rejection episodes in interim from last visit, patient survival, graft survival and kidney disease and disease status; chemistry panel; tacrolimus trough; and other immunosuppressant levels.

Post Transplant Month 36

For all patients, the following were completed: assessment of rejection episodes in interim from last visit, patient survival, graft survival and kidney disease and disease status; chemistry panel; tacrolimus trough; other immunosuppressant levels; BFXM, TFXM for routine management (samples to Local [optional] and Central Laboratories [mandatory]); DSA by luminex labScreen (sample to Central Laboratory only); and kidney allograft biopsy (duplicate slides to Central Pathology Laboratory).

Treatment of Persistent DSA Levels

DSA was analyzed both by central and local laboratory during treatment, at the end of the treatment period (Week 9) and at Months 3, 6, 12 and 36. Central Laboratory results at week 9 only was provided to the local centers. If the recipient maintains a positive DSA and a positive BFXM and/or TFXM as measured by the central laboratory (week 9 result) then plasmapheresis and/or intravenous immune globulin may be used to lower the DSA as follows: plasmapheresis and/or intravenous immune globulin was administered per the clinical judgment of the principal investigator.

Supplementary eculizumab as a booster following plasmapheresis/before FFP may be administered during weeks 9-10 only.

Other medications such as rituximab and bortezomib are not allowed to treat persistent DSA.

Serum samples were submitted to the central lab for DSA and B/TFXM testing. Serum samples were obtained prior to beginning plasmapheresis, then weekly during plasmapheresis (pre-plasmapheresis sample) and one month following the conclusion of plasmapheresis therapy.

Note: Eculizumab supplementation was not allowed for treatment of persistent DSA that extended beyond the 10^{th} postoperative week.

Treatment with Fresh Frozen Plasma

If a patient received FFP not associated with plasmapheresis then the patients receiving eculizumab would receive a supplemental dose of eculizumab (600 mg) 1 hour prior to FFP administration.

Early Discontinuations

Screen Failure

Patients who did not meet the study criteria during the screening/enrollment phase were considered screen failures. These patients were discontinued from the study without follow-up. A discontinuation case report form that documents the reason for the screening failure were completed.

Premature Discontinuations and Withdrawals

Early Termination Withdrawals or Discontinuations:

Reasons for early discontinuation or withdrawal were documented completely in the appropriate case report form.

If a patient discontinued the eculizumab study drug at any time during the study, the patient had additional study visits to ensure safety follow-up every 2 weeks for 2 months (maximum of 4 visits) following the final dose. These visits coincided with routine follow-up visits for maintenance of their kidney transplant per the transplant center. The last visit included all assessments listed for the Month 12 visit in the Schedule of Assessments (Table 5).

Abbreviated Physical Exam included vitals and weight; evaluation to assess transplant which included collections of blood samples; review of any changes in the patients' health; and appropriate study procedures if the patient was diagnosed with AMR during evaluation.

TABLE 5
Schedule of Assessment - Pre-Transplant Phase

| | Pre-Transplant Study Visit |
|---|---|
| | Screening and Enrollment |
| | Study Week |
| | Day −1 to Day 0 Prior to |
| | Transplant |
| | Visit Window |
| Procedure | N/A |
| Informed Consent | X |
| Demographics | X |
| Medical History | X |
| Physical Exam including | X |
| Vital Signs, Height and | |
| Weight | |
| Assessment of | X |
| Inclusion/Exclusion | |
| Conformity | |
| ECG | X |
| Vaccination against N. meningitidis^{b} | X |
| Provide Patient Safety | X |
| Card for N. meninqitidis | |
| Chemistry Panel including | X |
| SCr and BUN | |
| Hematology Panel including | X |
| WBC diff., Plts, Hgb | |
| Urinalysis | X |
| aPTT, PT and INR | X |
| Serum Pregnancy Test for | X |
| Women of Childbearing | |
| Potential | |
| BFXM and TFXM | X^{c} |
| CDC | X^{d} |
| DSA by Luminex LabScreen | X^{d} |
| Enrollment | X |
| Concomitant Medications | X |
| Adverse Event Assessment | X |

^{a}Abbreviated physical examination consisted of a body system relevant examination based upon Investigator judgment and patient symptoms.
^{b}Patients were vaccinated at least 14 days prior to receiving the first dose of eculizumab or be vaccinated and receive prophylactic treatment with an appropriate antibiotic for 14 days after the vaccination. Furthermore, all patients not already vaccinated within the time period of active coverage specified by the vaccine manufacturer, must be re-vaccinated 30 days after initial vaccination.
^{c}BFXM and TFXM levels were run at the Local Laboratory (optional). Duplicate samples were sent to the Central Laboratory for the Screening and Day −1 samples. The Local Laboratory specimens were used to select patients for study eligibility and determine if the patient can proceed to transplantation. Duplicate samples were sent to the Central Laboratory for confirmation. At all other interim time points selected by the Investigative Site for patient management, the Local Laboratory were used for processing of specimens. These interim samples did not need to be sent to the Central Laboratory.
^{d}CDC and/or DSA levels were to be run at the Local Laboratory. Duplicate samples were sent to the Central Laboratory for the Screening and Day −1 samples. The Local Laboratory specimens were used to select patients for study eligibility and determine if the patient could proceed to transplantation. Duplicate samples were sent to the Central Laboratory for confirmation. At all other interim time points selected by the Investigative Site for patient management, the Local Laboratory were used for processing of specimens. These interim samples did not need to be sent to the Central Laboratory.

TABLE 6
Schedule of Assessment - Immediate Post Transplant Phase

| Transplant Study | Transplant, | | | | | | | |
|---|---|---|---|---|---|---|---|---|
| Visit | Day 0 | Day 1 | Day 2 | Day 3 | Day 4 | Day 5 | Day 6 | Day 7 |
| Study Week | Week 0 | Week 0 | | | | | | Week 1 |
| Visit Window | 0 | 0 | 0 | 0 | 0 | 0 | 0 | 0 |
| Procedure | | | | | | | | |
| Kidney | X | | | | | | | |
| Transplantation | | | | | | | | |
| Physical Exam | X | | | | | | | |
| including Vital | | | | | | | | |
| Signs and Weight | | | | | | | | |
| Abbreviated | | X | X | X | X | X | X | X |
| Physical Exam | | | | | | | | |
| including Vital | | | | | | | | |
| Signs and Weight^{a} | | | | | | | | |
| Clinical | | X | X | X | X | X | X | X |
| Assessment | | | | | | | | |
| including | | | | | | | | |
| Evaluation for | | | | | | | | |
| Rejection | | | | | | | | |
| Administer | X^{c} | X^{d} | | | | | | X^{d} |
| Eculizumab^{b} | | | | | | | | |
| Hematology Panel | X | X | X | X | X | X | X | X |
| including WBC | | | | | | | | |
| diff., Plts, Hgb | | | | | | | | |
| Chemistry Panel | X | X | X | X | X | X | X | X |
| including SCr and | | | | | | | | |
| BUN | | | | | | | | |
| PK, PT and PD^{e} | B/P | T/P | | | | | | T/P |
| Urinalysis | X | | | | | | | X |
| Spot Urine for | | | | | | | | X |
| Urine | | | | | | | | |
| Protein/Creatinine | | | | | | | | |
| Ratio | | | | | | | | |
| aPTT and INR | X | X | X | X | X | X | X | X |
| Tacrolimus trough | | X | X | X | X | X | X | X |
| BFXM and TFXM^{f} | X | X | | | | | | X |
| DSA by Luminex | X | X | | | | | | X |
| LabScreen^{g} | | | | | | | | |
| Assess renal | X | X | X | X | X | X | X | X |
| function/need for | | | | | | | | |
| dialysis | | | | | | | | |
| Kidney Allograft | X | | | | | | | |
| Biopsy (post | | | | | | | | |
| reperfusion) | | | | | | | | |
| Concomitant | X | X | X | X | X | X | X | X |
| Medications | | | | | | | | |
| Immunosuppressive | X | X | X | X | X | X | X | X |
| Medications | | | | | | | | |
| Adverse Event | X | X | X | X | X | X | X | X |
| Assessment | | | | | | | | |

^{a}Abbreviated physical examination consisted of a body system relevant examination based upon Investigator judgment and patient symptoms.
^{b}No PP or IVIg were administered during the first 9 weeks unless biopsy-proven AMR
^{c}Administered eculizumab 1200 mg (4 vials) IV over 25-45 minutes one hour prior to re-perfusion of kidney
^{d}Administered eculizumab 900 mg (3 vials) IV on Days 1 and 7 post-transplantation over 25-45 minutes
^{e}B = Baseline sample; T = Trough sample; P = Peak sample. Baseline and trough samples for PK/PD were to be taken 5-90 minutes before the study drug infusion. Peak samples for PK/PD testing were to be taken 60 minutes after the completion of the study drug infusion.
^{f}BFXM and TFXM levels were drawn on Days 0, 1, and 7 and run at the Local Laboratory (optional). Duplicate samples were sent to the Central Laboratory. At all other interim time points selected by the Investigative Site for patient management, the Local Laboratory were used for processing of specimens. These interim samples did not need to be sent to the Central Laboratory. Local Laboratory specimen data were used for all patient management.
^{g}DSA levels were drawn on Days 0, 1, and 7 and run at the Local Laboratory. Duplicate samples were sent to the Central Laboratory. At all other interim time points selected by the Investigative Site for patient management, the Local Laboratory were used for processing of specimens. These interim samples did not need to be sent to the central laboratory. Local Laboratory specimen data were used for all patient management.

TABLE 7
Schedule of Assessment - Extended Post Transplant Phase

| | | | | Days | | | | | | | | Mo. | |
|---|---|---|---|---|---|---|---|---|---|---|---|---|---|
| Transplant Study | | Day | Day | 35 & | Day | Day | | Mo. | | Mo. | | 10 & | |
| Visit | Day 14 | 21 | 28 | 49 | 56 | 63 | Mo. 3 | 4 & 5 | Mo. 6 | 7 & 8 | Mo. 9 | 11 | Mo. 12 |
| Study Week | Week 2 | Week 3 | Week 4 | Week | Week 8 | Week 9 | Week | Week | Week | Week | Week | Week | Week |
| | | | | 5 & 7 | | | 12 | 17 & | 26 | 30 & | 38 | 44 & | 52 |
| | | | | | | | | 21 | | 34 | | 48 | |
| Visit Window | ±2 days | ±2 | ±2 | ±2 | ±2 | ±2 | ±7 | ±7 | ±7 | ±7 | ±7 | ±7 | ±7 |
| | | days | days | days | days | days | days | days | days | days | days | days | days |
| Procedure | | | | | | | | | | | | | |
| Physical Exam | | | | | | | X | | X | | | | X |
| Including Vital | | | | | | | | | | | | | |
| Signs and Weight | | | | | | | | | | | | | |
| Abbreviated | X | X | X | X | X | X | | X | | X | X | X | |
| Physical Exam | | | | | | | | | | | | | |
| Including Vital | | | | | | | | | | | | | |
| Signs and Weight^{a} | | | | | | | | | | | | | |
| Clinical | X | X | X | X | X | X | X | X | X | X | X | X | X |
| Assessment | | | | | | | | | | | | | |
| including | | | | | | | | | | | | | |
| Evaluation for | | | | | | | | | | | | | |
| Rejection | | | | | | | | | | | | | |
| Administer | X^{c} | X^{c} | X^{c} | X^{d} | | X^{d} | | | | | | | |
| Eculizumab^{b} | | | | | | | | | | | | | |
| Hematology Panel | X | X | X | | X | X | X | | X | | X | | X |
| including WBC | | | | | | | | | | | | | |
| diff, Plts, Hgb | | | | | | | | | | | | | |
| Chemistry Panel | X | X | X | X^{e} | X | X | X | X^{e} | X | X^{e} | X | X^{e} | X |
| including SCr and | | | | | | | | | | | | | |
| BUN | | | | | | | | | | | | | |
| PK and PD^{f} | T/P | | T/P | T/P | | T/P | | | | | | | |
| Urinalysis | | | X | | | X | X | | X | | | | X |
| Spot Urine for | | | X | | | X | X | | X | | | | X |
| Urine | | | | | | | | | | | | | |
| Protein/Creatinine | | | | | | | | | | | | | |
| Ratio | | | | | | | | | | | | | |
| aPTT, PT and INR | X | X | X | | X | X | X | | X | | X | | X |
| Tacrolimus trough | X | X | X | X | X | X | X | X | X | X | X | X | X |
| BFXM and TFXM^{g} | X | X | X | | | X | X | | X | | | | X |
| DSA by Luminex | X | X | X | | | X | X | | X | | | | X |
| LabScreen^{f} | | | | | | | | | | | | | |
| Assess Renal | X | X | X | X | X | X | X | X | X | X | X | X | X |
| Function/need | | | | | | | | | | | | | |
| for dialysis | | | | | | | | | | | | | |
| eGFR (MDRD 7) | | | X | | X | X | X | | X | | X | | X |
| Kidney Allograft | X | | | | | | X | | | | | | X |
| Biopsy | | | | | | | | | | | | | |
| Concomitant | X | X | X | X | X | X | X | X | X | X | X | X | X |
| Medications | | | | | | | | | | | | | |
| Immunosuppressive | X | X | X | X | X | X | X | X | X | X | X | X | X |
| Medications | | | | | | | | | | | | | |
| Adverse Event | X | X | X | X | X | X | X | X | X | X | X | X | X |
| Assessment | | | | | | | | | | | | | |

^{a}Abbreviated physical examination consisted of a body system relevant examination based upon Investigator judgment and patient symptoms.
^{b}No prophylactic PP or IVIg were administered during first 9 weeks unless biopsy-proven AMR
^{c}administered eculizumab 900 mg (3 vials) IV on Days 14, 21, 28 over 25-45 minutes
^{d}administered eculizumab 1200 mg (4 vials) IV at Weeks 5, 7, 9 over 25-45 minutes
^{e}SCr and BUN only.
^{f}T = Trough sample; P = Peak sample. Trough samples for PK/PD were to be taken 5-90 minutes before the study drug infusion. Peak samples for PK/PD testing were taken 60 minutes after the completion of the study drug infusion.
^{g}BFXM and TFXM levels were monitored on Days 14, 21, 28, Week 9 and Months 3, 6 and 12 and were run at the Local Laboratory (optional). Duplicate samples were sent to Central Laboratory. At all other interim time points selected by the Investigative Site for patient management, the Local Laboratory was used for processing of specimens. These interim samples did not need to be sent to the Central Laboratory. See Study Manual for sample processing information. Local Laboratory specimen data was used for all patient management.
^{f}DSA levels were monitored on Days 14, 21, 28, Week 9 and Months 3, 6 and 12 at the Local Laboratory. Duplicate samples were to be sent to Central Laboratory. At all other interim time points selected by the Investigative Site for patient management, the Local Laboratory was used for processing of specimens. These interim samples did not need to be sent to the Central Laboratory. Local Laboratory specimen data were used for all patient management.

TABLE 8
Schedule of Assessment - Long Term Outcome Data Collection

| | Post-Transplant Study Visit | | |
|---|---|---|---|
| | Month 18 | Month 24 | Month 36 |
| | Study Week | | |
| | Week 78 | Week 104 | Week 156 |
| | Vsit Window | | |
| Procedure | ±14 day | ±14 day | ±14 day |
| Assessments for Interim | X | X | X |
| Rejection Episodes, Graft | | | |
| Loss, Patient Survival, | | | |
| Kidney Disease and Disease | | | |
| Status^{a} | | | |
| Chemistry Panel including SCr | X | X | X |
| and BUN | | | |
| Tacrolimus Trough | X | X | X |
| Other Immunosuppresant Levels | X | X | X |
| BFXM and TFXM^{b} | | | X |
| DSA by Luminex LabScreen^{b} | | | X |
| Kidney Allograft Biospy^{c} | | | X |

^{a}Interim rejection episodes were recorded from previous visit through subsequent visit.
^{b}BFKM, TEXM, and DSA specimens were sent to the Central Laboratory only. See Study Manual for sample processing information.
^{c}Duplicate slides were sent to Central Pathology Laboratory.

### Selection and Withdrawal of Patients

All patients adhered to the following inclusion/exclusion criteria.

Patient Inclusion Criteria

(1) Male or female patients at least 18 years old. (2) Patients with Stage V chronic kidney disease who received a kidney transplant from a deceased donor to whom they were sensitized. (3) history of prior exposure to HLA: (a) prior solid organ or tissue allograft; (b) pregnancy; (c) blood transfusion; (d) prior exposure to specific donor's HLA; (4) historical positive CDC cross match and/or BFXM or TFXM from about 300 and to about 500 mcs (no patient may have a BFXM or TFXM greater than about 500 mcs, and/or DSA identified by single antigen bead (SAB) assay (Luminex Labscreen assay) with a single MFI greater than about 3000. (5) negative CDC at time of transplantation. (6) Able to understand the ICF and willing to comply with study procedures. (7) Female patients of child-bearing potential had a negative pregnancy test (serum beta-hCG) and must be practicing an effective, reliable and medically approved contraceptive regimen while on eculizumab treatment and for up to 5 months following discontinuation of treatment

Patient Exclusion Criteria.

(1) Had received treatment with eculizumab at any time prior to enrolling in this study. (2) ABO blood type incompatible with deceased donor. (3) history of severe cardiac disease (e.g. New York Heart Association [NYHA] Functional Class III or IV, myocardial infarction less than about 6 months of enrollment, ventricular tachyarrhythmias requiring ongoing treatment, unstable angina or other significant cardiovascular diseases). (4) prior splenectomy. (5) had a known bleeding disorder; (6) has any active bacterial or other infection that is clinically significant in the opinion of the Investigator and is a contraindication to transplantation; (7) had participated in any other investigational drug study or was exposed to an investigational drug or device within 30 days of screening; (8) had received rituximab (Mabthera®) 3 months prior to screening; (9) had received bortezomib (Velcade®) 3 months prior to screening; (10) had received alemtuzumab (Campath®) 6 months prior to screening; (11) hypersensitivity to murine proteins or to one of the product excipients; (12) history of illicit drug use or alcohol abuse within the previous year; (13) unresolved meningococcal disease; (14) pregnancy or Lactation; (15) current cancer or a history of cancer within the 5 years prior to screening, with the exception of patients who had successfully treated nonmetastatic basal or squamous cell carcinoma of the skin; carcinoma in situ of the cervix; or breast carcinoma in situ; (16) any medical condition that, in the opinion of the Investigator, might interfere with the patient's participation in the study, poses an added risk for the patient, or confounds the assessment of the patient; and (17) active infection with Hepatitis B (HBV), Hepatitis C (HCV) or Human Immunodeficiency Virus (HIV)

Patient Withdrawal Criteria

Patients will be informed that they had the right to withdraw from the study at any time for any reason without prejudice to their medical care.

Patients must be withdrawn from the study for any of the following reasons: (1) Patient request; (2) Patient is unwilling or unable to comply with the protocol; and;

The reasons for patient study drug and/or patient withdrawal must be recorded in the patient's case report form and in the source records. The Investigator must notify Alexion Pharmaceuticals and the Medical Monitor immediately when a patient has been discontinued/withdrawn due to an adverse event. All patients who are withdrawn from the study should complete the tests and evaluations scheduled for the final visit of the study.

If a patient was discontinued due to an adverse event, the event will be followed until it is resolved or in the opinion of the Principal Investigator the patient is determined to be medically stable. Every effort will be made to undertake protocol-specified safety follow-up procedures.

Patients who failed to return for final assessments were contacted by the site study staff in an attempt to have them comply with the protocol. As it was vital to obtain follow-up data on any patient withdrawn because of an adverse event or a serious adverse event, follow-up due diligence documentation consisted of 2 phone calls followed by 1 registered letter to the patient's last known address. In any case, every effort was made to undertake protocol-specified safety follow-up procedures.

Treatment of Patients

Description of Study Drug

Eculizumab is a recombinant humanized monoclonal IgG2/4κ antibody produced by murine myeloma cell culture and purified by standard bioprocess technology. Eculizumab contains human constant regions from human IgG2 sequences and human IgG4 sequences and murine complementarity-determining regions grafted onto the human framework light- and heavy-chain variable regions. Eculizumab is composed of two 448 amino acid heavy chains and two 214 amino acid light chains and has a molecular weight of approximately 148 kDa.

Post-Transplant Immunosuppression and Concomitant Medications

Patients who were enrolled and received their kidney transplants were required to take immunosuppressive and prophylactic medications to maintain allograft function and protect them from infection. In addition, medications were used to manage co-morbid conditions such as hypertension, hyperlipidemia, diabetes, and pain. These conditions were managed according to the standard of care practices at the individual investigative sites.

Among the medications that were given to transplant recipients were: Induction Therapy: Thymoglobulin (1.5 mg/kg×4 doses [6 mg/kg recommended, may use up to 7.5 mg/kg]); Maintenance Immunosuppression: Tacrolimus; maintain trough levels at 4 toll ng/mL for Months 1 through 12. No calcineurin inhibitor avoidance or withdrawal protocols are allowed. Mycophenolate mofetil (MMF; Cellcept®)/Enteric-coated mycophenolic acid (EC-MPA; Myfortic®); MMF: 1 gram BID (may titrate down or alter dosing schedule for patient intolerance); EC-MPA: 720 mg BID (may titrate down or alter dosing schedule for patient intolerance); Generic formulations of the above were acceptable for purposes of the study; Prednisone initially per standard of care at the transplant center and tapered to 5 mg daily by 3 months post-transplantation; No steroid avoidance or withdrawal protocols allowed.

Concomitant and Prophylactic Medications:

All concomitant medications were administered to all patients according to standard institutional protocols and applied uniformly to all patients. Examples of these medications included but were not restricted to: (a) prophylaxis; (b)Pneumocystis carinii/jiroveci Pneumonia (PCP) prophylaxis and; (c) Antifungal prophylaxis.

Induction, maintenance immunosuppression, and prophylactic therapies were used uniformly across all centers in the study and recorded in the case report form.

Prohibited Medications/Treatments

The following medications/treatments were prohibited as their use may compromise the findings or interact with eculizumab: (1) Use of alemtuzumab (Campath®) ≤6 months prior to screening and post-transplantation during the study; (2) Use of basiliximab (Simulect®) induction therapy during the study; (3) Use of bortezomib (Velcade®) ≤3 months prior to screening and post-transplantation during the study; Bortezomib was used at the discretion of the principal investigator for salvage therapy of AMR not responsive to first line therapy; (4) Use of rituximab (Mabthera®) ≤3 months prior to screening and post-transplantation during the study. Rituximab was used at the discretion of the principal investigator for salvage therapy of AMR not responsive to first line therapy; (5) Use of immunoadsorption at any time (in place of plasmapheresis) and; (6) Use of prophylactic plasmapheresis or intravenous immune globulin during the first 9 weeks post-transplantation during eculizumab treatment

DSA and Cell-Based Crossmatch Evaluations

Patients underwent routine post-transplantation monitoring for circulating DSA and cell-based cross-match evaluations as follows:

Per protocol clinical monitoring of DSA (Luminex LabScreen) and cell-based crossmatches included BFXM and TFXM will be performed by the central laboratory at Days 0, 1, 7, 14, 21, 28, Week 9, and Months 3, 6 and 12;

DSA, BFXM and TFXM tests were also collected at Month 36, but were not included in the primary efficacy analysis. They were sent to the Central Laboratory and used for purposes of long term follow up only.

Duplicate samples were sent to the transplant center's Local Laboratory for DSA and/or cell-based crossmatches to facilitate patient management. The Central Laboratory data was not be used for patient management.

Interim samples for patient management was analyzed at the transplant center's HLA Local Laboratory and may include any of the following tests: DSA, CDC, BFXM, and TFXM. Duplicate samples were not required for the Central Laboratory.

Treatment Compliance

Patients were administered eculizumab IV in a controlled setting such as a hospital, outpatient clinic or short-stay care unit, thereby ensuring compliance with study drug administration under the supervision of the Investigator. Study coordinators at the investigative sites ensured that all study participants were adequately informed on the specific treatment regimens required for compliance with the study protocol.

The sponsor or its designee periodically monitored study sites to ensure compliance with the protocol and communicated with sites on a regular basis regarding study protocol deviations. All protocol deviations were appropriately documented by the Investigator and study monitors.

Study Drug Materials and Management

Study Drug

Eculizumab was supplied in 30 mL vials with a solution concentration of 10 mg/mL. Each single entry 30 mL vial contained a solution concentration of 10 mg/mL and had enough solution to withdraw the indicated 30 mL.

Study Drug Packaging and Labeling

The study drug eculizumab was released to the site upon receipt of all required essential documents based upon federal, state, and local regulations. Each kit had a single panel label describing the contents and a place for the pharmacist to record the patient number and initials. The pharmacy immediately notified the distributor if vials were damaged. Eculizumab was stored in a secure, limited-access storage area.

Study Drug Storage

The study drug (eculizumab) vials were stored in the original carton until time of use under refrigerated conditions at 2-8° C. (36-46° F.) and protected from light. Eculizumab was not used beyond the expiration date stamped on the carton. Refer to below for stability and storage of diluted solutions of eculizumab. ECULIZUMAB WAS NOT FROZEN AND WAS NOT SHAKEN.

Study Drug Preparation

Infusions of the study drug was prepared using aseptic technique. Each vial of eculizumab contained 300 mg of active ingredient in 30 mL of product solution. Eculizumab was diluted to a final admixture concentration of 5 mg/mL using the following steps: withdraw the required amount of eculizumab from the vial into a sterile syringe; transfer the recommended dose to an infusion bag; diluted eculizumab to a final concentration of 5 mg/mL by adding the appropriate amount (equal volume of diluent to drug volume) of 0.9% Sodium Chloride Injection, USP; 0.45% Sodium Chloride Injection, USP; 5% Dextrose in Water Injection, USP; or Ringer's Injection, USP to the infusion bag. The final admixed eculizumab 5 mg/mL infusion volume was 120 mL for 600 mg doses, 180 mL for 900 mg doses or 240 mL for 1200 mg doses.

TABLE 8
Eculizumab and Diluent Volumes

| Eculizumab | | Volume of | | Volume of | | Total Volume of | |
|---|---|---|---|---|---|---|---|
| Dose | | Eculizumab | | Diluent ¹ | | Administration | |
| 600 | mg | 60 mL | (2 vials) | 60 | mL | 120 | mL |
| 900 | mg | 90 mL | (3 vials) | 90 | mL | 180 | mL |
| 1200 | mg | 120 mL | (4 vials) | 120 | mL | 240 | mL |

¹ One of the following diluents was used: a. 0.9% sodium chloride; b. 0.45% sodium chloride; c. 5% dextrose in water; d. Ringer's injection.

The infusion bag containing the diluted eculizumab was gently inverted solution to ensure thorough mixing of the product and diluent. Empty vials and vials with residual materials were kept for inspection by the study monitor prior to their destruction, or handled per local site pharmacy standard operating procedures for clinical study drugs. Prior to administration, the admixture was allowed to adjust to room temperature (18-25° C., 64-77° F.). The admixture was not heated in a microwave or with any heat source other than ambient air temperature. The eculizumab admixture was inspected visually for particulate matter and discoloration prior to administration.

Administration and Stability of Solution

Eculizumab was not Administered as an Intravenous Push or Bolus Injection.

The admixture was administered by IV infusion over 35 minutes (range 25-45 minutes). It was not necessary to protect the infusion bags from light while study drug was being administered to the patient. At the site's discretion, the diluted study drug was administered via gravity feed, a syringe-type pump, or an infusion pump. The patients were monitored for 1 hour following infusion.

Admixed solutions of eculizumab was stable for 24 hours at 2-8° C. (36-46° F.) and at room temperature. If the eculizumab was prepared more than 4 hours in advance of a patient's visit, the diluted material was stored at 2° C. to 8° C.

If an adverse event occurred during the administration of the study drug, the infusion was slowed or stopped at the discretion of the Investigator, depending upon the nature and severity of the event. The adverse event was captured in the patient's source document and case report form.

Study Drug Accountability

The current International Conference on Harmonization (ICH) Good Clinical Practice (GCP) Guidelines required the Principal Investigator to ensure that study drug deliveries from the Sponsor was received by a responsible person (e.g. pharmacist). In addition, the following guidelines were also adhered to: (1) Study drug deliveries were recorded; (2) Study drug was handled and stored safely and properly; (3) Study drug was only dispensed to patients in accordance with the protocol; (4) Unused study drug was returned to the Sponsor or standard procedures for the alternative disposition of unused study drug was followed.

When a study drug shipment was received at the site, the pharmacist verified the contents, signed the packing invoice provided with the shipment, and maintained the original copy for review by the study monitor. A signed copy was faxed to the contact provided on the packing list and the duplicate copy kept in the pharmacy binder.

Accountability logs and Inventory logs were provided to assist the pharmacist in maintaining current and accurate inventory records covering receipt, dispensing, and disposition of the study drug. During the study, the following information was noted in the accountability log: the patient number(s), initials of patient(s) to whom drug was dispensed, kit number, the date(s) and time that the study drug was prepared and dispensed, and the initials of the pharmacist or designee who prepared the study drug. Sites kept a running total of their drug supply. Empty vials and vials with residual materials were kept for inspection by the study monitor prior to their destruction, or handled per local site pharmacy standard operating procedures for clinical study drugs.

The study monitor examined the inventory during the study. Additionally, the inventory records were readily available and subject to regulatory authorities, the local regulatory agency, or an independent auditor's inspection at any time.

Study Drug Handling and Disposal

Drug inventory and accountability records for the study drug were kept by the Investigator/Pharmacist. Study drug accountability throughout the study was documented. The following guidelines were followed: (1) The Investigator agreed not to supply study drugs to any person except the patients of the study; (2) The Investigator/Pharmacist will keep the study drug in a pharmacy or other locked and secure storage facility under controlled storage conditions, accessible only to those authorized by the Investigator to dispense the investigative drug.

A study drug inventory was maintained by the Investigator/Pharmacist. The inventory included details of material received and a clear record of when they were dispensed, and to which patient.

At the conclusion or termination of this study, the Investigator/Pharmacist agreed to conduct a final drug supply inventory and to record the results of this inventory on the drug accountability record. Delivery records and records of used or returned study drug was reconcilable. The person responsible at the investigative site signed appropriate forms for deliveries and returns.

Used or unused study drug were destroyed at the study center according to standard institutional procedures after the Sponsor or designee had conducted drug accountability. A copy of the standard institutional procedure for destroying investigational drugs was provided to the Sponsor or designee upon request.

Unused study drug not destroyed at the site was returned to the Sponsor or designee at the end of the study or upon expiration.

Assessment of Efficacy

Kidney Allograft Biopsy Evaluations

For cause allograft biopsies were obtained for clinical signs of allograft dysfunction based upon at least one of the following criteria, with or without elevation of DSA from baseline (day of transplant): (1) A decrease in serum creatinine less than 10% per day in three consecutive days in the first week post transplantation compared to Day 0 immediate post-transplantation creatinine; (2) An increase in serum creatinine of ≥30% from nadir. Nadir was defined as the lowest serum creatinine within the first week post-transplantation; (3) Oliguria; (4) Clinical suspicion of AMR.

For-cause kidney biopsy slides were read at the transplant center and used for clinical management. Slides that were read locally were sent to the Central Pathology Laboratory for review.

Protocol Biopsy—Mandated biopsies were performed: (1) Post reperfusion (Intra-operative); (2) Day 14 post-transplantation; (3) Month 3 post-transplantation; (4) Month 12 post-transplantation; (5) Month 36 post-transplantation (for long term follow up only; will not be included in primary efficacy analysis).

For-cause kidney biopsies were required to confirm the diagnosis of AMR. Protocol biopsies were used to monitor subclinical changes in the allograft. These were performed to assist in the diagnosis of subclinical instances of AMR that were only evidenced histologically.

Protocol kidney biopsies were used to evaluate other secondary endpoints and also for evaluation of subclinical cases of AMR that were only evident on a histological basis. Protocol biopsies were read at the transplant center and were used for clinical management. Slides that were read locally were sent to the Central Pathology Laboratory.

Treatment of AMR Episodes

The cumulative incidences of AMR at Week 9 and through Month 12 of the study were the primary and secondary endpoints respectively. Should it occur, the following guidelines were used in the treatment of AMR.

For AMR Occurring During the Treatment Period Post-Transplantation

If the patient had a biopsy-proven diagnosis (from local pathologist) of clinically significant (elevated creatinine) AMR during the first 9 weeks post-transplantation, the patient were considered a treatment failure. AMR episodes were treated according to local standard of care protocols and at the Principal Investigators' discretion (with the exception of prohibited medications).

If the patient received plasmapheresis for the treatment of AMR and it was determined by the Principal Investigator that the patient remained on eculizumab, then supplemental doses of eculizumab were used as follows: eculizumab 600 mg (2 vials) were administered within 1 hour of completing each plasmapheresis session.

This was in order to maintain levels between 50 and 100 μg/mL of eculizumab, as had been predicted based on empirical experience and pharmacokinetics and pharmacodynamics modeling calculations for eculizumab under conditions of plasmapheresis.

Doses were given IV over 25-45 minutes.

AMR were treated with eculizumab for at least 5-weeks or until the serum creatinine returned to within 104 of their pre-rejection baseline creatinine or until they achieved a new stable baseline serum creatinine defined as less than a 20% variation on three successive tests taken at least 24 hours apart. The maximum number of weeks that the patient were treated with eculizumab for acute AMR was 9.

For AMR Occurring After the Week 9 Treatment Period

AMR episodes occurring after Week 9 were treated according to local standard of care protocols and at the Principal Investigators' discretion (with the exception of prohibited medications). Eculizumab was used to treat diagnosed AMR. See herein for general administration guidelines. If eculizumab was used to treat AMR, dosing was as follows (weeks are calculated from the day of first dose of eculizumab after AMR diagnosis): initial dose 900 mg (Day 1), if dosed within 7 days of last dose of eculizumab; initial dose 1200 mg (Day 1), if dosed after 7 days of last dose of eculizumab; 900 mg weekly for 4 doses (Weeks 1, 2, 3 and 4; ±2 days), then; 1200 mg every other week beginning on Week 5 for Weeks 5, 7, and 9 (±2 days)

AMR was treated with eculizumab for at least 5 weeks or until the serum creatinine returned to within 104 of their pre-rejection baseline creatinine or until they achieved a new stable baseline serum creatinine defined as less than a 20% variation on three successive tests taken at least 24 hours apart. The maximum number of weeks that the patient was treated with eculizumab for acute AMR was 9.

Assessment of Safety

Data Monitoring Committee

An independent data monitoring committee was comprised of at least 3 clinicians experienced in high-risk kidney transplantation. Other members also had expertise in the following areas: nephrology transplant specialist and/or transplant surgeon, infectious disease, and biostatistics. Since its primary function was to ensure patient safety, the data monitoring committee had access to all safety data, and a data management expert was part of the data monitoring committee to ensure timely delivery of all required data. The data monitoring committee also had access to a statistician and/or an epidemiologist if necessary.

The broad remit of the data monitoring committee was to monitor safety and efficacy data as it is accumulated and to make decisions on study conduct and dose regimen to ensure patients' safety. The operational details and responsibilities of the data monitoring committee was specified in a charter.

Safety Parameters

Demographic/Medical History

The demographic information to be collected included date of birth, gender, race and/or ethnicity.

Medical history information was collected includes all ongoing conditions and relevant/significant medical history (including all major hospitalizations and surgeries). Symptoms related to renal transplantation and/or the underlying etiology of the disease listed on the medical history form. Worsening of any of these signs or symptoms during the course of this study was captured as an adverse event.

Vital Signs

The following vital signs were collected: body temperature (° C.), heart rate (beats/min), respiratory rate (breaths/min), and blood pressure (mmHg).

Weight and Height

Height (cm) and weight (kg) were collected at screening. Post screening visits included weight collection only.

Physical Examinations

A complete physical exam consisted of an examination of the following: general Appearance, skin, head, ears, eyes, nose and throat (HEENT), cardiovascular, pulmonary, abdomen/gastrointestinal, neurological, lymph nodes, spine, extremities, and musculoskeletal. A genitourinary examination was performed unless a separate examination was performed within 1 year by another physician and is documented in the patient record.

Abbreviated physical exams were completed at the time points specified on the schedule of assessments. The body systems included in these exams were based on Investigator judgment and/or patient symptoms.

Electrocardiogram

A 12-lead ECG were performed. The data collected include includes heart rate, PR, QRS and QT intervals (uncorrected) and any abnormalities.

Laboratory Assessments

Hematology

The hematology panel was include complete blood count (CBC), with differential and platelet counts. CBC includes red blood cells (RBCs), white blood cells (WBCs), hemoglobin, hematocrit, mean corpuscular volume (MCV), mean corpuscular hemoglobin (MCH) and mean corpuscular hemoglobin concentration (MCHC).

Blood Chemistry Panel

The blood chemistries included: sodium, potassium, carbon dioxide, chloride, blood urea nitrogen, creatinine, glucose, calcium, magnesium, phosphorus, alkaline phosphatase, alanine aminotransferase (ALT), aspartate aminotransferase (AST), gamma-glutamyl transferase (GGT), lactic dehydrogenase (LDH), total and direct bilirubin, total protein, albumin, uric acid, and total cholesterol.

Coagulation

The coagulation testing included an activated partial thromboplastin time (aPTT), prothrombin time (PT) and international normalized ratio (INR).

Urinalysis

Urinalysis testing included protein, glucose, ketones, occult blood, and WBCs by dipstick, with microscopic examination and spot urine for urine protein/creatinine ratio.

Pregnancy Screen

At screening, a pregnancy test (serum beta-hCG) was completed for all females of child bearing potential.

Adverse and Serious Adverse Events

Adverse Event

An adverse event was defined as any untoward medical occurrence in a patient enrolled into this study regardless of its causal relationship to study treatment. Patients were instructed to contact the Principal Investigator or Sub-Investigator if any symptoms developed at any time after the informed consent and informed assent (if applicable) had been signed. If there was any doubt as to whether or not a clinical observation was an adverse event, the event was recorded and reported.

A treatment-emergent adverse event was defined as any event not present prior to exposure to Investigational Product or any event already present that worsens in either intensity or frequency following exposure to Investigational Product.

Adverse events were assigned Medical Dictionary for Regulatory Activities (MedDRA) preferred terms and tabulated as incidence rates per treatment group.

Safety evaluations consisted of monitoring and recording all adverse events, including serious adverse events, the regular monitoring of hematology, blood chemistry and urine results. In addition, regular monitoring of vital signs, physical condition and body weight measurements were performed.

The safety reference document for this clinical trial was the Investigator brochure.

Serious Adverse Event

A serious adverse event was an adverse event occurring during any study phase (i.e., baseline, treatment, or follow-up), and at any dose of the investigational product that fulfills one or more of the following: (1) results in death; (2) it was immediately life-threatening; the term “life-threatening” meant that the patient was at risk of death at the time of the event. It did not refer to an event which hypothetically might have caused death if it were more severe; (3) it required in-patient hospitalization or prolongation of existing hospitalization. It results in persistent or significant disability or incapacity; (4) Results in a congenital abnormality or birth defect.

Important medical events that may not result in death, be life-threatening, or require hospitalization were considered a serious adverse events when, based upon appropriate medical judgment, they jeopardized the patient and required medical or surgical intervention to prevent one of the outcomes listed in this definition. Examples of such medical events included allergic bronchospasm requiring intensive treatment in an emergency room or at home, blood dyscrasias or convulsions that did not result in patient hospitalization or the development of drug dependency or drug abuse.

All serious adverse events that occur after any patient had been enrolled, before treatment, during treatment, or throughout the duration of the patient follow-up, whether or not they were related to the study, were recorded.

Other Adverse Events of Interest

Other adverse events of interest were identified by the Drug Safety Physician and if applicable also by the Clinical Study Team Physician during the evaluation of safety data for the Clinical Study Report. Significant adverse events of particular clinical importance, other than serious adverse events and those adverse events leading to discontinuation of the patient from the study, were classified as other adverse evens of interest. For each other adverse event of interest, a narrative was written and included in the Clinical Study Report.

Other Adverse Events of Interest for this study included: (1) cumulative incidence of clinically significant infection (confirmed by culture, biopsy, genomic, or serologic findings) that required hospitalization or anti-infective treatment, or was otherwise deemed significant by the Investigator; (2) cumulative incidence of CMV disease; (3) cumulative incidence of BK virus disease; (4) cumulative incidence of encapsulated bacterial infection; (5) cumulative incidence of PTLD (post-transplant lymphoproliferative disease); (6) cumulative incidence of malignancy; (7) cumulative incidence of biopsy-proven acute cellular rejection; (8)proportion of patients that develop severe acute cellular rejection that do not respond to thymoglobulin or other lymphocyte depleting agents; (9) cumulative incidence of allograft loss for reasons other than AMR; (10) overall patient survival.

Relationship to Study Drug

An Investigator who was qualified in medicine must make the determination of relationship to the investigational product for each adverse event (Unrelated, Unlikely, Possible, Probable, or Definite).

Unrelated: This relationship suggested that there was no association between the Investigational Product and the reported event.

Unlikely: This relationship suggested that the clinical picture was highly consistent with a cause other than the Investigational Product but attribution could not be made with absolute certainty and a relationship between the Investigational Product and AE could not be excluded with complete confidence.

Possible: This relationship suggested that treatment with the Investigational Product caused or contributed to the adverse events, i.e. the event followed a reasonable temporal sequence from the time of drug administration and/or followed a known response pattern to the Investigational Product, but also could have been produced by other factors.

Probable: This relationship suggests that a reasonable temporal sequence of the event with the Investigational Product administration existed and the likely association of the event with the Investigational Product. This was based upon the known pharmacological action of the Investigational Product, known or previously reported adverse reactions to the Investigational Product or class of drugs, or judgment based on the Principal Investigator's clinical experience.

Definite: Temporal relationship to the Investigational Product, other conditions (concurrent illness, concurrent medication reaction, or progression/expression of disease state) did not appear to explain event, corresponds with the known pharmaceutical profile, improvement on discontinuation, re-appearance on re-challenge.

Recording Adverse Events

Adverse events spontaneously reported by the patient and/or in response to an open question from the study personnel or revealed by observation were recorded during the study at the investigational site as per the timetable listed in Tables 7, 8, and 9. Clinically significant changes in laboratory values, blood pressure, and pulse were reported as adverse events. Abnormal values that constituted serious adverse events or lead to discontinuation of administration of study drug were reported and recorded as an adverse events. Information about adverse events were collected from the signing of the informed consent form until the end of the study. Serious adverse events information were collected from signing of informed consent form until the end of the study. The medical term for the adverse event was reported in standard medical terminology when possible. For each adverse event, the Investigator evaluated and reported the onset (date and time), resolution (date and time), intensity, causality, action taken, serious outcome (if applicable), and whether or not it caused the patient to discontinue the study.

Intensity was assessed according to the following scale: (1) Mild (awareness of sign or symptom, but easily tolerated); (2) Moderate (discomfort sufficient to cause interference with normal activities); (3) Severe (incapacitating, with inability to perform normal activities and may require systemic drug therapy or other treatment).

It was important to distinguish between serious and severe adverse events. Severity was a measure of intensity whereas seriousness was as defined by the criteria herein. An adverse event of severe intensity may not be considered serious.

If it became known during the administration of the study drug that a patient is pregnant, the study drug was stopped immediately. In addition, for any woman who became pregnant at any time during the study, Pharmacovigilance was notified via the same method as serious adverse event reporting. Pharmacovigilance supplied the Investigator with a copy of a “Pregnancy Reporting and Outcome Form”. The patient was followed until the outcome of the pregnancy was known (spontaneous miscarriage, elective termination, normal birth or congenital abnormality), even if the patient was discontinued from the study. When the outcome of the pregnancy becomes known the form completed and returned to Pharmacovigilance. If additional follow-up was required, the Investigator was requested to provide the information.

Pregnancy in itself was not regarded as an adverse event unless there was a suspicion that an investigational product may have interfered with the effectiveness of a contraceptive medication.

All reports of congenital abnormalities/birth defects were serious adverse events. Spontaneous miscarriages were also reported and handled as serious adverse events. Elective abortions without complications were not handled as adverse events.

Reporting Adverse Events

The Investigator was responsible for reporting all adverse events and serious adverse events observed or reported during the study regardless of their relationship to the study drug or their clinical significance.

All adverse events that occurred after the patient was given consent was reported in detail in the patient's source/chart and on the appropriate case report form and followed to satisfactory resolution or until the Principal Investigator or Sub-Investigator deemed the event to be chronic or the patient to be stable. The description of the adverse event include the onset date, resolution date, intensity, seriousness, and the likelihood of relationship of the adverse event to the study drug.

Additional information to be reported included any required treatment or evaluations, and outcome. All reported adverse events were followed to adequate resolution. Any medical condition that was present at the time that the patient was screened but did not deteriorate was reported as an adverse event. However, if it deteriorated at any time during the study and this deterioration was felt to be related to study drug, it was recorded as an adverse event.

All serious adverse events (related and unrelated) were recorded from the signing of consent form until the end of the study. All serious adverse events were reported to Pharmacovigilance Designee within one business day of the first awareness of the event. Additionally, any serious adverse events considered possibly or probably related to the investigational product and discovered by the Investigator at any time after the study was reported. The Investigator completed, signed and dated the serious adverse event pages, verify the accuracy of the information recorded on the serious adverse event pages with the corresponding source documents.

Additional follow-up information, if required or available, was faxed to Pharmacovigilance Designee within one business day of receipt and this was completed on a follow-up serious adverse event form and placed with the original serious adverse event information and kept with the appropriate section of the case report form and/or study file.

The Principal Investigator was responsible for notifying the relevant regulatory authorities of certain events. It was the Principal Investigator's responsibility to notify the institutional review board or independent ethics committee of all serious adverse events that occurred at his or her site per their local institutional review board or independent ethics committee established guidelines for submission. Investigators were notified of all unexpected, serious, drug-related events (7/15 Day Safety Reports) that occurred during the clinical study. Each site was responsible for notifying its institutional review board or independent ethics committee of these additional serious adverse events.

Statistics and Data Analysis

General Considerations for Data Analysis

Details of the statistical analysis described below was specified in a separate Statistical Analysis Plan prior to data lock and analysis. Any deviations from the statistical plan was specified and justified in the Clinical Study Report.

For continuous data, the mean, standard deviation, median, minimum and maximum was reported. For categorical data, percent and frequency was reported.

Missing Data

Missing data on demographic, recipient-, donor- and transplant-related information and on laboratory data was treated as missing; no method for imputation was planned. Missing data on time to event endpoints had events coded as right censored per the following table:

TABLE 9
Missing Data Events Coding for Time to Event
Data Analyses

| Endpoint | Right Censoring |
|---|---|
| Time to First | Patients who did not experience a biopsy- |
| Biopsy-proven AMR | proven AMR at any time during follow-up will |
| | be right censored as of the date of last |
| | patient contact. |
| Time to First | Patients who did not experience a biopsy- |
| Biopsy-proven ACR | proven ACR at any time during follow-up will |
| | be right censored as of the date of last |
| | patient contact. |
| Graft Survival | Patients who are alive with functioning graft |
| | will be right censored as of the date of last |
| | patient contact. |
| Patient Survival | Patients who are still alive as of the last |
| | known follow-up will be right censored as of |
| | the date of last patient contact. |

Analysis Datasets

Full Analysis Set

Patients who were enrolled, received a deceased donor kidney transplant, and received at least one dose of eculizumab was included in the full analysis set. All efficacy analyses was performed using the full analysis set.

Per Protocol Set

Patients who experienced a major protocol deviation that was deemed to have affected outcome was excluded from the full analysis set to create the Per Protocol analysis set. Efficacy analyses was only performed using the Per Protocol set if the percent of patients in the Per Protocol set compared to the full analysis set was less than 80%. The Per Protocol set was determined and documented prior to database lock.

Safety Set

Patients who were enrolled and received at least one dose of eculizumab were included in the Safety set. All safety analyses were performed using the Safety set.

Efficacy Analysis

The primary analysis of all endpoints occurred after all patients had reached Month 12 post-transplantation. Patients continued to be followed on Months 18, 24 and 36 for collection of additional follow up data on patient and graft survival, kidney disease and disease status.

Primary Efficacy Variable and Analysis

The diagnosis of AMR was based on kidney allograft dysfunction and biopsy performed “for cause.” The histological diagnosis was based on Banff 2007 criteria for AMR as determined by the Central Pathology Laboratory. For this study only level II and level III AMR were accepted as defined below:

Presence of circulating anti-DSAs antibodies, morphologic evidence of acute tissue injury, such as (Type/Grade): Banff 2007 Level II—Capillary and/or glomerular inflammation (ptc/g>0) and/or thromboses; and Banff 2007 Level III—Arterial-v3;

The primary efficacy variable was a binary outcome variable where patients meeting the above composite endpoint definition were considered treatment failures and all others were considered treatment successes. The point estimate of the incidence of treatment failure at 9 weeks post-transplantation was calculated along with an exact 95% confidence interval. The null hypothesis that the true rate of treatment failure at 9 weeks post-transplantation was equal to 40% was tested using the exact binomial test.

Secondary Efficacy Variables and Analyses

Secondary efficacy endpoints included: cumulative incidence of AMR that occurs between Week 9 and Month 12 post-transplantation (AMR of any grade that meets Banff 2007 criteria); (1) Treatment failure rate defined as the occurrence of 1) biopsy-proven AMR, 2) graft loss, 3) patient death, 4) loss to follow up at Month 12 post-transplantation; (2) graft and patient survival at Months 6 and 12 post-transplantation; (3) histological evidence of AMR on protocol biopsies without other clinical findings at Day 14, and Months 3 and 12 post-transplantation; (4) overall pathological changes, including chronic AMR, on protocol biopsies at Day 14, and Months 3 and 12 post-transplantation; (5) cumulative number of plasmapheresis treatments at 12 months post-transplantation; (6) cumulative incidence of patients requiring splenectomy at 12 months post-transplantation; (7) incidence of DGF post-transplantation (defined as the requirement for dialysis within the first post-transplantation week for reasons other than post-operative hyperkalemia, acute pulmonary edema or fluid overload due to comorbid conditions); (8) cumulative incidence and duration of dialysis between 7 days and 12 months post-transplantation; (9) number of days the serum creatinine is more than 30% above nadir following the diagnosis of AMR; (10) stable renal function between Week 4 and Month 12 post-transplantation as measured by: (a) Estimated Glomerular Filtration Rate (calculated) MDRD7 on at least 3 consecutive measurements taken at least 2 days apart while not on plasmapheresis or dialysis that vary ≤20%; (b) serum creatinine defined as the value on at least 3 consecutive measurements taken at least 2 days apart while not on plasmapheresis or dialysis that vary ≤20%; (15) patient and graft survival, the cumulative incidence of delayed AMR, the cumulative incidence of biopsy-proven AMR without other clinical findings, and the cumulative incidence of biopsy-proven acute cellular rejection, each at the times post-transplantation listed above, will be estimated using the product-limit (Kaplan-Meier) method. In addition to point estimates, 95% CIs will be provided; (17) the incidence of treatment of AMR diagnosed solely on histological evidence on protocol biopsies will be provided along with 95% confidence intervals. The actual treatments used will be summarized or listed; (18) the percentage of patients requiring splenectomy, the incidence of DGF, and the incidence of dialysis beyond 7 days post-transplantation will be provided along with 95% confidence intervals; (19) the duration of dialysis beyond 7 days post-transplantation, and the number of days that serum creatinine is more than 30% above nadir following the diagnosis of AMR summarized using descriptive statistics, and (20) overall pathological changes on protocol biopsies at Day 14, and Months 3 and 12, and change in renal function between Week 4 and Month 12, will be summarized using descriptive statistics.

Safety Analysis

Safety assessments consisted of summarizing all adverse events, including serious adverse events, hematology, blood chemistry and urine results, regular monitoring of vital signs, physical condition and body weight measurements.

All adverse events (serious and non-serious), regardless of relationship to study drug, were classified by system organ class and preferred term using the Medical Dictionary for Regulatory Activities (version 10.1 or higher). Incidence rates were tabulated for each system organ class and preferred term.

In addition to the above, the following specific safety assessments were summarized for the study at week 9 and month 12 post transplantation: (1) cumulative incidence of clinically significant infection (confirmed by culture, biopsy, genomic or serologic findings) that required hospitalization or anti-infective treatment, or was otherwise deemed significant by the Investigator; (2) cumulative incidence of CMV disease (incidence and %); (3) cumulative incidence of BK virus disease (incidence and %); (4) cumulative incidence of encapsulated bacterial infections (incidence and %); (5) cumulative incidence of PTLD; (6) cumulative incidence of malignancy; (7) cumulative incidence of biopsy-proven acute cellular rejection of any grade that meets Banff 2007 criteria; (8)proportion of patients that develop severe acute cellular rejection that do not respond to thymoglobulin or other lymphocyte depleting agents; (9) cumulative incidence of allograft loss for reason other than AMR; and (10) overall patient survival.

Interim Analysis

No formal statistical interim analyses of the primary and secondary efficacy variables were planned.

Long Term Outcomes Data Collection

For purposes of long term follow up data collection to evaluate interim rejection episodes, graft loss, patient survival, kidney disease and disease status, all patients will be seen at Months 18, 24, and 36. The following information will be collected: Chemistry panel (including BUN and sCr); tacrolimus trough levels; other immunosuppressive levels; DSA, BFXM and TFXM (Month 36 only); and Kidney allograft biopsy (Month 36 only).

These data were not considered as part of the primary efficacy analysis.

Sample Size and Power Considerations

The primary efficacy composite endpoint is the Week 9 post-transplantation treatment failure rate defined as the occurrence of 1) biopsy-proven AMR, 2) graft loss, 3) patient death, or 4) loss to follow-up. Sample size and power considerations were based on the primary efficacy variable and a single-arm study with the following assumptions:

Composite endpoint true treatment failure rate at Week 9 post-transplantation with standard of care in the study population is, π₀=40%

Composite endpoint treatment failure rate at Week 9 post-transplantation with eculizumab is, π₁=20%

Null hypothesis, H₀: π₁=40%

Alternative hypothesis, H₁: π₁#40%

Type I error, α=0.05 (two-sided significance test)

Statistical test=Exact binomial test

An exact binomial test with a nominal 0.050 two-sided significance level will have >90% power to detect a difference between the null hypothesis proportion, π₁ of 0.400 and the alternative proportion, π₁, of 0.200 when the sample size is 80.

Outcomes

Twelve-month follow-up data based showed that eculizumab was effective in reducing the incidence of aAMR in deceased-donor kidney transplant recipients who were sensitized to their donors. Moreover, the rate of graft survival, patient survival, and kidney function at 1 year were similar to those expected for nonsensitized kidney transplant recipients.

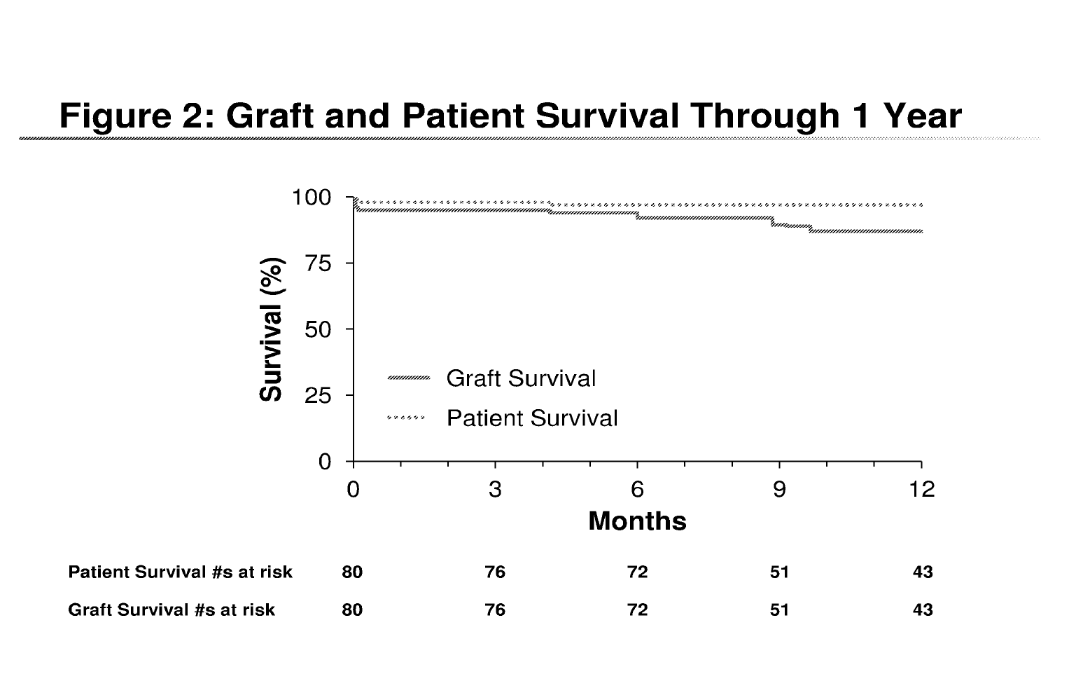

These results are summarized in tables 10 to 13 and FIG. 2.

TABLE 10
Baseline Demographics and Clinical Characteristics

| Characteristic | Patients (N = 80) | |
|---|---|---|
| Median age, y (range) | 52 | (24-70) |
| Time on waiting list^{a}, y (range) | 5.5 | (0.3-33.6) |
| Sex, n (%) (male/female) | 32 (40)/48 (60) | |
| Current DSA^{b}, n (%) | 69 | (86.3) |
| Class I only, n (%) | 30 | (37.5) |
| Class II only, n (%) | 12 | (15.0) |
| Class I and II, n (%) | 27 | (33.8) |
| Historical^{c} DSA, n (%) | 11 | (13.8) |

^{a}Time from start of chronic dialysis to first dose of eculizumab
^{b}Qualifying DSA by SAP ≥3000 MFI on current serum on the day of transplant.
^{c}Qualifying DSA by SAP ≥3000 MFI and positive CDC crossmatch on historical serum, negative on current serum on the day of transplantation.
CDC, complement-dependent cytotoxicity;
DSA, donor-specific antibody;
MFI, mean fluorescence intensity;
SAB, single-antigen bead;
SD, standard deviation.

TABLE 11
Efficacy Endpoints

| | 9 Weeks | 1 Year |
|---|---|---|
| Outcome | (N = 80) | (N = 80) |
| Post-transplant failure rate,^{a} n | 10 (12.5) | 15 (18.8) |
| (%) | (95% CI: 6.2%, | (95% CI: 10.9%, |
| | 21.8%) | 29.0%) |
| Biopsy-proven AMR, n (%) | 6 (7.5) | 8 (10.0) |
| Graft loss, n (%) | 4 (5.0) | 9 (11.3) |
| Primary cause | 2 (2.5) | 2 (2.5) |
| Renal artery thrombosis | 2 (2.5) | 2 (2.5) |
| Primary nonfunction | | 2 (2.5) |
| Acute rejection | 1 (1.3) | 2 (2.5) |
| Chronic rejection | 0 | 1 (1.3) |
| Death with functioning | | 2 (2.5) |
| allograft | | 6 (7.5) |
| Death, n (%) | | |
| Lost to follow-up, n (%) | | |

Data from local laboratory instead of the central laboratory.
28 of 80 patients had neither a treatment failure nor the full 12 months of follow-up as of the time of the data transfer.
^{a}Composite endpoint including: (1) biopsy-proven AMR (grade II or III), (2) graft loss, (3) patient death, and/or (4) loss to follow-up.
AMR, antibody-mediated rejection

TABLE 12
Patients with For-cause Grade 2 or 3 aAMR in First 12 Months

| Time Period | Patients, n (%)(N = 80) |
|---|---|
| Total at 1 year | 8 (10.0) |
| 0-9 weeks | 6 (7.5) |
| 9 weeks-6 months | 2 (2.5) |
| 6 months-12 months | 0 |

TABLE 13
Patients with All Rejection Types by Time of First Occurrence

| Type of Rejection, Time Period | Patients (N = 80) | For Cause |
|---|---|---|
| Acute AMR | 22 | 13 |
| <9 weeks | 14 | 9 |
| 9 wk-6 m | 7 | 4 |
| 6 m-12 m | 1 | 0 |
| Chronic AMR | 2 | 0 |
| <9 weeks | 0 | 0 |
| 9 w-6 m | 0 | 0 |
| 6 m-12 m | 2 | 0 |
| Acute CMR | 5 | 2 |
| <9 weeks | 1 | 1 |
| 9 w-6 m | 4 | 1 |
| 6 m-12 m | 0 | 0 |
| Chronic CMR | 0 | 0 |

### Example 2: Efficacy and Safety of Eculizumab for Treatment of AMR

This is an open-label analysis that will compare eculizumab versus plasmapheresis (PP) and immunoglobulin (IVIg) for the treatment of AMR in renal transplant recipients. All patients will be evaluated from the time of AMR diagnosis for 12 months.

The primary efficacy measure will be the percent change in estimated glomerular filtration rate at 3 months post-treatment.

Conclusions

Eculizumab is effective at reducing the estimated glomerular filtration by at least 20% at 3 months post-treatment in adult renal transplant recipients who develop AMR.

Study Design

Allocation: Randomized; Endpoint Classification: Safety/Efficacy Study; Intervention Model: Parallel Assignment; Masking: Open Label; Primary Purpose: Treatment

Condition

AMR; Humoral Rejection

Intervention

Drug: Eculizumab; Other Name: Soliris; Biological: Immunoglobulin; Other Name: IVIg;

Study Arm (s)

Active Comparator: Standard of Care; Plasmapheresis (PP)×3, at 40-60 cc/kg; Immunoglobulin (IVIg), to be administered after each PP; Interventions: Biological: Immunoglobulin; Procedure: Plasmapheresis; Experimental: Soliris (eculizumab); 1200 mg first dose (Time: Screening/Week “0”, after Biopsy Proven AMR); 900 mg weekly for 4 doses (Weeks 1, 2, 3, 4); 1200 mg week 5; Week 6: If DSAs are less than 50% of baseline DSA then no further treatment, otherwise 1200 mg weeks 7, 9; Intervention: Drug: Eculizumab

Recruiting: Twenty-one patients.

Eligibility

Inclusion Criteria:

1. Adult renal transplant recipients, men and women between 18 and 75 years of age; 2. Any patient with acute graft dysfunction (elevation of creatinine above post transplant nadir) and, two out of three, of the following Inclusion Criteria: (a) presence of circulating anti HLA antibody (DSA); (b) histological findings compatible with Banff Class II or III AMR on transplant biopsy; and (c) peritubular capillary c4d positivity on transplant biopsy.

Exclusion Criteria:

1. patients that had received eculizumab prior to enrolling in the study; 2. patients that had ongoing non-acute AMR; 3. patients with predominantly chronic AMR or interstitial fibrosis/tubular atrophy; 4. history of severe cardiac disease (e.g., New York Heart Association [NYHA] Functional Class III or IV, myocardial infarction 6 months of randomization, ventricular tachyarrhythmias requiring ongoing treatment, unstable angina or other significant cardiovascular diseases); 5. prior splenectomy; 6. had a known bleeding disorder; 7. had any active bacterial or other infection which was clinically significant in the opinion of the Investigator and was a contraindication to transplantation; 8. had participated in any other investigational drug study or was exposed to an investigational drug or device within 30 days of screening; 9. had received rituximab (Rituxan®) ≤3 months prior to screening; 10. had received bortezomib (Velcade@) ≤3 months prior to screening; 11. had received alemtuzumab (Campath®) ≤6 months prior to screening; 12. needed concurrent treatment with anti thymocyte globulin (Thymoglobulin®); 13. had hypersensitivity to murine proteins or to one of the product excipients; 14. history of illicit drug use or alcohol abuse within the previous year; 15. unresolved meningococcal disease; 16. pregnancy or lactation; 17. current cancer or a history of cancer within the 5 years prior to screening with the exception of patients who have successfully treated non-metastatic basal or squamous cell carcinoma of the skin; carcinoma in situ of the cervix; or breast carcinoma in situ; 18. any medical condition that, in the opinion of the Investigator, may have interfered with the patient's participation in the study, posed an added risk for the patient, or confounded the assessment of the patient active infection with Hepatitis B (HBV), Hepatitis C (HCV) or human immunodeficiency virus (HIV).

### APPENDICES

### Appendix A

Banff Criteria 2007 Update^{[52]}

### Solez et al.

TABLE 3
Banff 97 diagnostic categories for renal allograft biopsies-Banff‘07 update ^{1, 2}

| | |
|---|---|
| 1. | Normal |
| 2. | Antibody-mediated changes (may coincide with categories 3, 4 and 5 and 6) |
| | Due to documentation of circulating antidonor antibody, and C4d³ or allograft pathology |
| | C4d deposition without morphologic evidence of active rejection |
| | C4d+, presence of circulating antidonor antibodies, no signs of acute or chronic TCMR or ABMR (i.e. g0, cg0, ptc0, no ptc |
| | lamination). Cases with simultaneous borderline changes or ATN are considered as indeterminate |
| | Acute antibody-mediated rejection⁴ |
| | C4d+, presence of circulating antidonor antibodies, morphologic evidence of acute tissue injury, such as (Type/Grade): |
| | I. ATN-like minimal inflammation |
| | II. Capillary and or glomerular inflammation (ptc/g > 0) and/or thromboses |
| | III. Arterial-v3 |
| | Chronic active antibody-mediated rejection⁴ |
| | C4d+, presence of circulating antidonor antibodies, morphologic evidence of chronic tissue injury, such as glomerular double |
| | contours and/or peritubular capillary basement membrane multilayering and/or interstitial fibrosis/tubular atrophy and/or fibrous |
| | intimal thickening in arteries |
| 3. | Borderline changes: ‘Suspicious’ for acute T-cell-mediated rejection (may coincide with categories 2 and 5 and 6) |
| | This category is used when no intimal arteritis is present, but there are foci of tubulitis (t1, t2 or t3) with minor interstitial infiltration (i0 |
| | or i1) or interstitial infiltration (i2, i3) with mild (t1) tubulitis |
| 4. | T-cell-mediated rejection (TCMR, may coincide with categories 2 and 5 and 6) |
| | Acute T-cell-mediated rejection (Type/Grade:) |
| | IA. Cases with significant interstitial infiltration (>25% of parenchyma affected, i2 or i3) and foci of moderate tubulitis (t2) |
| | IB. Cases with significant interstitial infiltration (>25% of parenchyma affected, i2 or i3) and foci of severe tubulitis (t3) |
| | IIA. Cases with mild-to-moderate intimal arteritis (v1) |
| | IIB. Cases with severe intimal arteritis comprising >25% of the luminal area (v2) |
| | III. Cases with ‘transmural’ arteritis and/or arterial fibrinoid change and necrosis of medial smooth muscle cells with accompanying |
| | lymphocytic inflammation (v3) |
| | Chronic active T-cell-mediated rejection |
| | ‘chronic allograft arteriopathy’ (arterial intimal fibrosis with mononuclear cell infiltration in fibrosis, formation of neo-intima) |
| 5. | Interstitial fibrosis and tubular atrophy, no evidence of any specific etiology |
| | (may include nonspecific vascular and glomerular sclerosis, but severity graded by tubulointerstitial features) |
| | Grade |
| | I. Mild interstitial fibrosis and tubular atrophy (<25% of cortical area) |
| | II. Moderate interstitial fibrosis and tubular atrophy (26-50% of cortical area) |
| | III. Severe interstitial fibrosis and tubular atrophy/loss (>50% of cortical area) |
| 6. | Other: Changes not considered to be due to rejection-acute and/or chronic (for diagnoses see Table 14 in (42); may include isolated |
| | g, cg or cv lesions and coincide with categories 2, 3, 4 and 5) |

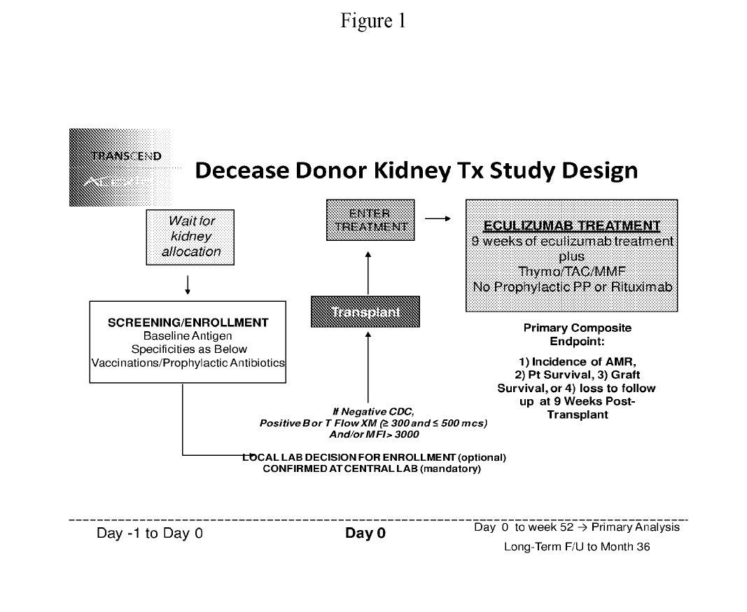

¹ The 2007 updates are underlined.
² All existing scoring categories (g, t, v, i, cg, ct, ci, cv, ah, mm) remain unchanged (42)
³Please refer to Table 2 and FIG. 1.
⁴Suspicious for antibody-mediated rejection if C4d (in the presence of antibody) or alloantibody (C4d+) not demonstrated in the presence of morphologic evidence of tissue injury.

MDRD 7 (Estimated GFR)

Modification of Diet in Renal Disease (MDRD) 7 Calculation [53]:

MDRD 7 equation (MDRD7)=170×[serum creatinine(mg/dL)]−0.999×[age]−0.176×[0.762 if patient is female]×[1.18 if patient is black]×[serum urea nitrogen concentration (mg/dL)]−0.170×[serum albumin concentration (g/dL)]0.318

List of Laboratory Tests

Chemistry, Coagulation, Hematology, Urinalysis, Pregnancy, and HLA Tests:

Chemistry

| | | | |
|---|---|---|---|
| Sodium | Carbon | Total | AST |
| | Dioxide | Cholesterol | |
| Potassium | Albumin | Total | ALT |
| | | Protein | |
| Chloride | BUN | Creatinine | Alkaline |
| | | | Phosphatase |
| Calcium | Magnesium | Phosphorus | Glucose |
| Uric Acid | LDH | GGT | Total and |
| | | | Direct |
| | | | Bilirubin |

Coagulation

| | | |
|---|---|---|
| aPTT | PT | INR |

Complete Blood Count with Differential and
Platelet Count

| | | | |
|---|---|---|---|
| Hemoglobin | Hematocrit | RBC | WBC |
| MCV (mean | Mean | Mean | Platelets |
| corpuscular | Corpuscular | Corpuscular | |
| volume) | Hemoglobin | Hemoglobin | |
| | (MCH) | Concentration | |
| | | (MCHC) | |

Urinalysis with Microscopy

| | | |
|---|---|---|
| Protein | Ketones | WBC's by |
| | | dipstick |
| Glucose | Occult Blood | Microscopy |

Spot Urine for Urine Protein/Creatinine Ratio
Pregnancy Testing (if applicable)
Serum beta-hCG

HLA Laboratory Testing:

Donor Specific Antibody Test - DSA
Complement Dependent Cytotoxicity - CDC
B-cell Flow Cross Match - BFXM
T-cell Flow Cross Match - TFXM

| [symbol] | | | |
|---|---|---|---|
| [symbol] | [symbol] | [symbol] | [symbol] |
| [symbol] | | | |
| [symbol] | | | |
| [symbol] | | | |
| [symbol] | | | |

| [symbol] | | |
|---|---|---|
| [symbol] | [symbol] | [symbol] |

| [symbol] | | | |
|---|---|---|---|
| [symbol] | [symbol] | [symbol] | [symbol] |
| [symbol] | | | |

| [symbol] | | |
|---|---|---|
| [symbol] | [symbol] | [symbol] |
| [symbol] | | |

[symbol]

[symbol]

[symbol]

[symbol]

indicates data missing or illegible when filed

[symbol]

[symbol]

[symbol]

[symbol]

[symbol]

[symbol]

indicates data missing or illegible when filed

### Appendix B: List of References

1. Takemoto, S. K., et al., National conference to assess antibody-mediated rejection in solid organ transplantation. Am J Transplant, 2004. 4(7): p. 1033-41.
2. McKenna, R. M., S. K. Takemoto, and P. I. Terasaki, Anti-HLA antibodies after solid organ transplantation. Transplantation, 2000. 69(3): p. 319-26.
3. Feucht, H. E. and G. Opelz, The humoral immune response towards HLA class II determinants in renal transplantation. Kidney Int, 1996. 50(5): p. 1464-75.
4. Mauiyyedi, S., et al., Acute humoral rejection in kidney transplantation: II. Morphology, immunopathology, and pathologic classification. J Am Soc Nephrol, 2002. 13(3): p. 779-87.
5. Singh, N., J. Pirsch, and M. Samaniego, Antibody-mediated rejection: treatment alternatives and outcomes. Transplant Rev (Orlando), 2009. 23(1): p. 34-46.
6. Trpkov, K., et al., Pathologic features of acute renal allograft rejection associated with donor-specific antibody, Analysis using the Banff grading schema. Transplantation, 1996. 61(11): p. 1586-92.
7. Collins, A. B., et al., Complement activation in acute humoral renal allograft rejection: diagnostic significance of C4d deposits in peritubular capillaries. J Am Soc Nephrol, 1999. 10(10): p. 2208-14.
8. Halloran, P. F., The clinical importance of alloantibody-mediated rejection. Am J Transplant, 2003. 3(6): p. 639-40.
9. Lefaucheur and Glotz, How to grade immunological risk using sensitive HLA donor specific antibodies detection techniques. Trends in Transplant 4, 2010.
10. Montgomery, R. A. and A. A. Zachary, Transplanting patients with a positive donor-specific crossmatch: a single center's perspective. Pediatr Transplant, 2004. 8(6): p. 535-42.
11. Thielke, J. J., et al., Living donor kidney transplantation across positive crossmatch: the University of Illinois at Chicago experience. Transplantation, 2009. 87(2): p. 268-73.
12. Truong, L. D., et al., Acute antibody-mediated rejection of renal transplant: pathogenetic and diagnostic considerations. Arch Pathol Lab Med, 2007. 131(8): p. 1200-8.
13. Stegall, M. D., et al., A comparison of plasmapheresis versus high-dose IVIG desensitization in renal allograft recipients with high levels of donor specific alloantibody. Am J Transplant, 2006. 6(2): p. 346-51.
14. Rostaing, L., C. Guilbeau-Frugier, and N. Kamar, Rituximab for humoral rejection after kidney transplantation: an update. Transplantation, 2009. 87(8): p. 1261.
15. Faguer, S., et al., Rituximab therapy for acute humoral rejection after kidney transplantation. Transplantation, 2007. 83(9): p. 1277-80.
16. Crespo, M., et al., Acute humoral rejection in renal allograft recipients: I. Incidence, serology and clinical characteristics. Transplantation, 2001. 71(5): p. 652-8.
17. White, N. B., et al., Successful rescue therapy with plasmapheresis and intravenous immunoglobulin for acute humoral renal transplant rejection. Transplantation, 2004. 78(5): p. 772-4.
18. Braun, N., et al., Successful treatment of accelerated vascular rejection in a highly immunised renal transplant recipient with immunoadsorption and 15-deoxyspergualin. Transpl Int, 2004. 17(7): p. 384-6.
19. Han, D. J., et al., Treatment and Prognosis of Late Onset Humoral Rejection in Comparison With Early Onset Humoral Rejection in Kidney Transplants: Abstract 526. Transplantation, 2008. 86(2S): p. 184.
20. Muro, M., et al., Acute vascular rejection mediated by HLA antibodies in a cadaveric kidney recipient: discrepancies between FlowPRA, ELISA and CDC vs luminex screening. Nephrol Dial Transplant, 2005. 20(1): p. 223-6.
21. Higgins, R., et al., The histological development of acute antibody-mediated rejection in HLA antibody-incompatible renal transplantation. Nephrol Dial Transplant, 2009. 25(4): p. 1306-12.
22. Montgomery, R. A., et al., Desensitization in HLA-incompatible kidney recipients and survival. N Engl J Med. 365(4): p. 318-26.
23. Stegall, M. D., et al., Terminal complement inhibition decreases antibody-mediated rejection in sensitized renal transplant recipients. Am J Transplant. 11(11): p. 2405-13.
24. Russell, J. D., et al., The quality of life in renal transplantation—a prospective study. Transplantation, 1992. 54(4): p. 656-60.
25. http://optn.transpiant.hrsa.gov.
26. Colvin, R. B., Antibody-mediated renal allograft rejection: diagnosis and pathogenesis. J Am Soc Nephrol, 2007. 18(4): p. 1046-56.
27. Burns, J. M., et al., Alloantibody levels and acute humoral rejection early after positive crossmatch kidney transplantation. Am J Transplant, 2008. 8(12): p. 2684-94.
28. Lederer, S. R., et al., Impact of humoral alloreactivity early after transplantation on the long-term survival of renal allografts. Kidney Int, 2001. 59(1): p. 334-41.
29. Haas, M., et al., Subclinical acute antibody-mediated rejection in positive crossmatch renal allografts. Am J Transplant, 2007. 7(3): p. 576-85.
30. Hillmen, P., et al., The complement inhibitor eculizumab in paroxysmal nocturnal hemoglobinuria. N Engl J Med, 2006. 355(12): p. 1233-43.
31. Richards, S. J., A. Hill, and P. Hillmen, Recent advances in the diagnosis, monitoring, and management of patients with paroxysmal nocturnal hemoglobinuria. Cytometry B Clin Cytom, 2007. 72(5): p. 291-8.
32. Nurnberger, J., et al., Eculizumab for atypical hemolytic-uremic syndrome. N Engl J Med, 2009. 360(5): p. 542-4.
33. Rother, R. P., et al., C5 blockade with conventional immunosuppression induces long-term graft survival in presensitized recipients. Am J Transplant, 2008. 8(6): p. 1129-42.
34. Wang, H., et al., Inhibition of terminal complement components in presensitized transplant recipients prevents antibody-mediated rejection leading to long-term graft survival and accommodation. J Immunol, 2007. 179(7): p. 4451-63.
35. Wang, H., et al., Prevention of acute vascular rejection by a functionally blocking anti-C5 monoclonal antibody combined with cyclosporine. Transplantation, 2005. 79(9): p. 1121-7.
36. GTR 56-Determination of Dissociation Constants for N19/8 and m5G1.1. 2004.
37. PAI IM 1184. Eculizumab Human Tissue Cross Reactivity Study. 2005.
38. GTR 55-Epitope Mapping of m5G1.1. 2004.
39. GTR 52-Cloning and Humanization of m5G1.1 to Create h5G1.1. 2004.
40. GTR-84-Genetic Engineering of h5G1.1-mAbs with either an IgG4 or Hybrid IgG2/G4 Human Heavy Constant Region. 2005.
41. GTR-104-Pharmacodynamics and Pharmacokinetics of C5-Deficient Mice Reconstituted with Human C5 and Treated with h5G1.1-mAb. 2005.
42. Stegall, M. D., et al., Abstract 178. American Transplant Congress 2009.
43. Locke, J. E., et al., The use of antibody to complement protein C5 for salvage treatment of severe antibody-mediated rejection. Am J Transplant, 2009. 9(1): p. 231-5.
44. Gloor, J. M., et al., Baseline donor-specific antibody levels and outcomes in positive crossmatch kidney transplantation. Am J Transplant, 2010. 10(3): p. 582-9.
45. Stegall, M. D., T. X. Diwan, and L. D. Cornell, Terminal complement inhibition decreases early acute humoral rejection in sensitized renal transplant recipients 2010: p. Abstract 020.01, XXIII meeting of The Transplantation Society, Vancouver, Canada.
46. Stegall, M., T. Diwan, and S. Raghavaiah, Terminal Complement Inhibition Decreases Antibody Mediated Rejection in Sensitized Renal Transplant Recipients. NEMJ, Submitted 2011.
47. Cornell, L. D., et al., Abstract 393. American Transplant Congress 2009.
48. Eculizumab Investigator's Brochure.
49. Eculizumab (Solirisa) Package Insert.
50. Schrezenmeier, H., et al., Abstract 3012. American Society of Hematology 2009.
51. Nankivell, B. J. and J. R. Chapman, The significance of subclinical rejection and the value of protocol biopsies. Am J Transplant, 2006. 6(9): p. 2006-12.
52. Solez, K., et al., Banff 07 classification of renal allograft pathology: updates and future directions. Am J Transplant, 2008. 8(4): p. 753-60.
53. Poge, U., et al., MDRD equations for estimation of GFR in renal transplant recipients. Am J Transplant, 2005. 5(6): p. 1306-11.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QWVLNTPLT
SEQ ID NO: 7
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCARY
FFGSSPNWYFDVWGQGTLVTVSS
SEQ ID NO: 8
DIQMTQSPSSLSASVGDRVTITCGASENIYGALNWYQQKPGKAPKLLIYGA
TNLADGVPSRFSGSGSGTDFTLTISSLQPEDFATYYCQNVLNTPLTFGQGTK
VEIK
SEQ ID NO: 9
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
SEQ ID NO: 10
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWMNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPV
TKSFNRGEC
SEQ ID NO: 12
QVQLVOGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSS ASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKC
CVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKV
SNKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVF
SCSVMHEALHNHYTQKSLSLSPGK
SEQ ID NO: 16
QVQLVQSGAEVKKPGASVKVSCKASGYISNYWIQWVRQAPGQGLEWM
GEILPGSGSTEYTENFKDRVTMIRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQGNVFSCSVMHEALHNHYTQKSLSLS
PGK
SEQ ID NO: 17
GASENIYEALN
SEQ ID NO: 18
EILPGSGHTEYTENFKD
SEQ ID NO: 19
GHIFSNYNIQ
SEQ ID NO: 20
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMIS
RTPEVTCVVVDVSQESPEVQFNWYVDGVEVHNAKTKPREEQFNSTYRVVSVL
TVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGGIGPFFGTANY
AQKFQGRVTITADESTSTAYMELSSLRSEDTAVYYCARDTPYFD YWGQGTLVTVSS
SEQ ID NO: 28
DIELTQPPSVSVAPGQTARISCSGDSIPNYYVYWYQQKPGQAPVLVIYDDSNRPSGIPERFSGSN
SGNTATLTISGTQAEDEADYYCQSFDSSLNAEVFGGGTK LTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPKGLEWMGIIDPDDSYTEYSPSFQ
GQVTI SADKSISTAYLQWSSLKASDTAMYYCARYEYGGFDI WGQGTLVTVSS
SEQ ID NO: 36
SYELTQPPSVSVAPGQTARISCSGDNIGNSYVHWYQQKPGQAPVLVIYKDNDRPSGIPERFSGSNS
NT ATLTISGTQAEDEADYYCGTYDIESYVFGGGTKLTV L

1. A method for preventing antibody mediated rejection in a human kidney transplant recipient comprising:
i. selecting a deceased donor;
ii. selecting a kidney transplant recipient, wherein the recipient is sensitized to the donor;
iii. transplanting the kidney from the donor to the recipient; and
iv. administering a therapeutically effective dose of an anti-C5 antibody, or binding fragment thereof to the recipient.

2. The method of claim 1, wherein the anti-C5 antibody, or binding fragment thereof reduces the likelihood that the recipient will develop antibody mediated rejection.

3. The method of claim 1, wherein the therapeutically effective dose of the anti-C5 antibody, or binding fragment thereof reduces the cumulative incidence of antibody mediated rejection that occurs between 9 weeks and 12 months post-transplantation.

4. The method of claim 1, wherein the therapeutically effective dose of the anti-C5 antibody, or binding fragment thereof reduces the treatment failure rate defined as the occurrence of: (a) biopsy proven AMR; (b) graft loss; (c) patient death; and (d) loss to follow up at 12 months post transplantation.

5. The method of claim 1, wherein the therapeutically effective dose of the anti-C5 antibody, or binding fragment thereof improves the graft and patient survival at months 6 and 12 months post-transplantation.

6. The method of claim 1, wherein the therapeutically effective dose of the anti-C5 antibody, or binding fragment thereof reduces:
(a) the cumulative number of plasmapheresis treatments at 12-months post-transplantation;
(b) the incidence of patients requiring splenectomy at 12-months post-transplantation;
(c) the cumulative incidence and duration of dialysis between 7 days and 12-months post-transplantation; and/or
(d) cumulative number of days the serum creatinine is more than 30% above its nadir following the diagnosis of antibody mediated rejection.

7-9. (canceled)

10. The method of claim 1, wherein the therapeutically effective dose of the anti-C5 antibody, or binding fragment thereof improves the renal function between 4 weeks and 12-months post-transplantation as measured by:
i. the estimated glomerular filtration rate calculated by Modification of Diet in Renal Disease 7 (MDRD7) on at least 3 consecutive measurements taken at least 2 days apart while not on plasmapheresis or dialysis that vary ≤20%, and
ii. serum creatinine defined as the value on at least 3 consecutive measurements that vary ≤20% taken at least 2 days apart while not on plasmapheresis or dialysis.

11. The method of claim 1, wherein the likelihood of developing antibody mediated rejection is reduced at 9 weeks, 12 months, 18 months, 24 months, 30 months, or 36 months post transplantation.

12-16. (canceled)

17. The method of claim 1, wherein the therapeutically effective dose comprises:
(a) a 1200 mg dose on the day of the transplant, and 900 mg of the anti-C5 antibody, or binding fragment thereof on the following post-transplantation days: day 1, 7, 14 (±2 days) and 21 (±2 days); and
(b) 1200 ma of the anti-C5 antibody, or binding fragment thereof on the following post-transplantation weeks: week 5 (±2 days), week 7 (±2 days) and week 9 (±2 days).

18. (canceled)

19. The method of claim 1, wherein on the day of the transplant the anti-C5 antibody, or binding fragment thereof is administered prior to reperfusion of the kidney allograft.

20. The method of claim 1, wherein the anti-C5 antibody, or binding fragment thereof is administered from about 30 minutes to about 3 hours prior to reperfusion of the kidney allograft r about 1 hour prior to reperfusion of the kidney allograft.

21. (canceled)

22. The method of claim 1, wherein the day 1 dose of the anti-C5 antibody, or binding fragment thereof is administered from about 18 to about 30 hours after reperfusion of the kidney allograft or about 24 hours after reperfusion of the kidney allograft.

23. (canceled)

24. The method of claim 1, wherein the anti-C5 antibody, or binding fragment thereof is maintained at plasma levels of about 50 to about 100 μg/mL.

25. The method of claim 1, wherein the recipient's medical history includes at least one sensitizing event selected from the group consisting of: prior solid organ or tissue allograft; pregnancy; blood transfusion; and prior exposure to the specific donor's HLA.

26. The method of claim 1, wherein the recipient has:
(a) a historical positive complement-dependent cvytotoxicity cross-match;
(b) a B cell flow cytometric cross-match from about 300 to about 500 mean channel shift;
(c) a T cell flow cytometric cross-match from about 300 to about 500 mean channel shift; and/or
(d) a donor specific antibody identified by a single antigen bead assay with a single mean fluorescence intensity greater than about 3000.

27-34. (canceled)

35. The method of claim 1, wherein the kidney allograft survives for at least six months, one year, three years, five years, or the remaining life of the recipient.

36-39. (canceled)

40. The method of claim 1, further comprising a step of administering at least one immunosuppressive drug selected from the group consisting of tacrolimus, mycophenolate mofetil, and prednisone.

41. (canceled)

42. A method for treating antibody mediated rejection in a kidney transplant recipient comprising the steps of:
i. selecting a kidney transplant recipient having symptoms of antibody mediated rejection;
ii. administering a therapeutically effective dose of an anti-C5 antibody or fragment thereof to the recipient;
wherein the dose of anti-C5 antibody, or fragment thereof reduces the symptoms of antibody mediated rejection in kidney transplant recipients.

43. The method of claim 42, wherein the therapeutically effective dose is a dosing schedule that comprises 1200 mg first dose; 900 mg weekly for 4 doses (Weeks 1, 2, 3, 4) and 1200 mg at week 5.

44. The method of claim 43, wherein the therapeutically effective dose further comprises a step of administering 1200 mg of the anti-C5 antibody or antigen-binding fragment at weeks 7 and 9.

45. The method of claim 42, further comprising a step of administering plasmapheresis and/or immunoglobulin to the recipient.

46-48. (canceled)

49. The method of claim 42, wherein the symptoms of antibody mediated rejection include acute graft dysfunction, (elevation of creatinine above post transplant nadir) and two out of three, of the following inclusion criteria:
i. presence of circulating donor specific antibodies;
ii. histological findings consistent with Banff Class II or III antibody mediated rejection on transplant biopsy; and
iii. peritubular capillary c4d positivity on transplant biopsy.

50. The method of claim 42, wherein the recipient has an increase in glomerular filtration rate at 3 months or 12 months post treatment.

51. (canceled)

52. The method of claim 42, wherein the anti-C5 antibody, or antigen binding fragment thereof, comprises:
(a) CDR1, CDR2, and CDR3 heavy chain sequences as set forth in SEQ ID NOs: 1, 2, and 3, respectively, and CDR1, CDR2, and CDR3 light chain sequences as set forth in SEQ ID NOs: 4, 5, and 6, respectively;
(b) a VH domain having the sequence set forth in SEQ ID NO:7, and a VL domain having the sequence set forth in SEQ ID NO:8, respectively;
(c) a heavy chain constant region having the amino acid sequences set forth in SEQ ID NO: 9; or
(d) heavy chain and light chains having the amino acid sequences set forth in SEQ ID NO: 10 and SEQ ID NO: 11, respectively;

53-55. (canceled)

56. The method of claim 42, wherein the anti-C5 antibody, or antigen binding fragment thereof, comprises:
(a) CDR1, CDR2, and CDR3 heavy chain sequences as set forth in SEQ ID NOs:19, 18, and 3, respectively, and CDR1, CDR2, and CDR3 light chain sequences as set forth in SEQ ID NOs: 4, 5, and 6, respectively;
(b) a VH domain having the sequence set forth in SEQ ID NO: 12, and a VL domain having the sequence set forth in SEQ ID NO:8, respectively;
(c) a heavy chain constant region having the amino acid sequences set forth in SEQ ID NO: 13; or
(d) heavy chain and light chains having the amino acid sequences set forth in SEQ ID NO: 14 and SEQ ID NO: 11, respectively.

57-59. (canceled)

60. The method of claim 42, wherein the anti-C5 antibody, or antigen binding fragment thereof comprises:
a) heavy chain and light chains having the amino acid sequences set forth in SEQ ID NO: 20 and SEQ ID NO: 11, respectively;
(b) CDR1, CDR2, and CDR3 heavy chain sequences as set forth in SEQ ID NOs:21, 22, and 23, respectively, and CDR1, CDR2, and CDR3 light chain sequences as set forth in SEQ ID NOs: 24, 25, and 26, respectively;
(c) VH domain having the sequence set forth in SEQ ID NO:27, and the VL domain having the sequence set forth in SEQ ID NO:2; or
(d) CDR1, CDR2, and CDR3 heavy chain sequences as set forth in SEQ ID NOs:29, 30, and 31, respectively, and CDR1, CDR2, and CDR3 light chain sequences as set forth in SEQ ID NOs: 32, 33, and 34, respectively.

61-63. (canceled)


SEQUENCE LISTING (36 sequences)

SEQ ID NO: 1 - protein, 10 aa, Artificial Sequence
  Description of Artificial Sequence Synthetic peptide
GYIFSNYWIQ

SEQ ID NO: 2 - protein, 17 aa, Artificial Sequence
  Description of Artificial Sequence Synthetic peptide
EILPGSGSTEYTENFKD

SEQ ID NO: 3 - protein, 13 aa, Artificial Sequence
  Description of Artificial Sequence Synthetic peptide
YFFGSSPNWYFDV

SEQ ID NO: 4 - protein, 11 aa, Artificial Sequence
  Description of Artificial Sequence Synthetic peptide
GASENIYGALN

SEQ ID NO: 5 - protein, 7 aa, Artificial Sequence
  Description of Artificial Sequence Synthetic peptide
GATNLAD

SEQ ID NO: 6 - protein, 9 aa, Artificial Sequence
  Description of Artificial Sequence Synthetic peptide
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Description of Artificial Sequence Synthetic peptide
GASENIYHALN

SEQ ID NO: 18 - protein, 17 aa, Artificial Sequence
  Description of Artificial Sequence Synthetic peptide
EILPGSGHTEYTENFKD

SEQ ID NO: 19 - protein, 10 aa, Artificial Sequence
  Description of Artificial Sequence Synthetic peptide
GHIFSNYWIQ

SEQ ID NO: 20 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  Description of Artificial Sequence Synthetic peptide
SYAIS

SEQ ID NO: 22 - protein, 17 aa, Artificial Sequence
  Description of Artificial Sequence Synthetic peptide
GIGPFFGTANYAQKFQG

SEQ ID NO: 23 - protein, 7 aa, Artificial Sequence
  Description of Artificial Sequence Synthetic peptide
DTPYFDY

SEQ ID NO: 24 - protein, 11 aa, Artificial Sequence
  Description of Artificial Sequence Synthetic peptide
SGDSIPNYYVY

SEQ ID NO: 25 - protein, 7 aa, Artificial Sequence
  Description of Artificial Sequence Synthetic peptide
DDSNRPS

SEQ ID NO: 26 - protein, 11 aa, Artificial Sequence
  Description of Artificial Sequence Synthetic peptide
QSFDSSLNAEV

SEQ ID NO: 27 - protein, 116 aa, Artificial Sequence
  Description of Artificial Sequence Synthetic polypeptide
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Description of Artificial Sequence Synthetic polypeptide
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  Description of Artificial Sequence Synthetic peptide
NYIS

SEQ ID NO: 30 - protein, 17 aa, Artificial Sequence
  Description of Artificial Sequence Synthetic peptide
IIDPDDSYTEYSPSFQG

SEQ ID NO: 31 - protein, 8 aa, Artificial Sequence
  Description of Artificial Sequence Synthetic peptide
YEYGGFDI

SEQ ID NO: 32 - protein, 11 aa, Artificial Sequence
  Description of Artificial Sequence Synthetic peptide
SGDNIGNSYVH

SEQ ID NO: 33 - protein, 7 aa, Artificial Sequence
  Description of Artificial Sequence Synthetic peptide
KDNDRPS

SEQ ID NO: 34 - protein, 9 aa, Artificial Sequence
  Description of Artificial Sequence Synthetic peptide
GTYDIESYV

SEQ ID NO: 35 - protein, 116 aa, Artificial Sequence
  Description of Artificial Sequence Synthetic polypeptide
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  Description of Artificial Sequence Synthetic polypeptide
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL